(19) United States
(12) Patent Application Publication
(10) Pub. No.: US 2006/0235024 A1
(43) Pub. Date: Oct. 19, 2006

(54) Acetylinic piperazine compounds and their use as metabotropic glutamate receptor antagonists

(75) Inventors: Chris Bryan, Mississauga (CA); Methvin Isaac, Mississauga (CA); Tomislav Stefanac, Mississauga (CA); Abdelmalik Slassi, Mississauga (CA); Louise Edwards, Mississauga (CA)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB; NPS Pharmaceuticals Inc.

(21) Appl. No.: 11/060,470

(22) Filed: Aug. 17, 2005

Related U.S. Application Data
(60) Provisional application No. 60/545,290, filed on Feb. 18, 2004.

(51) Int. Cl. A61K 31/496; A61K 31/495; C07D 409/02; C07D 241/04

(52) U.S. Cl. 514/252.13; 514/255.01; 544/379; 544/386

(57) ABSTRACT

The present invention relates to new acetylinic piperazine compounds of formula I, their pharmaceutically acceptable salts, and hydrates: 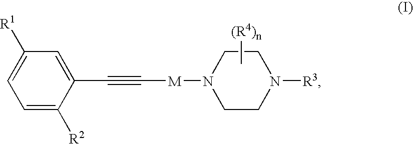
wherein R¹, R², R³, R⁴, M, and n are as defined in the description. The invention also relates to processes for the preparation of the compounds and to new intermediates employed in the preparation, pharmaceutical compositions containing the compounds, and to the use of the compounds in therapy.

### BACKGROUND OF THE INVENTION

The present invention relates to a new class of acetylinic piperazine compounds, to pharmaceutical compositions containing the compounds and to the use of the compounds in therapy. The present invention further relates to processes for the preparation of the compounds and to new intermediates used in the preparation thereof.

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999). Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5, and their alternatively spliced variants. The binding of agonists to these receptors results in the activation of phospholipase C and the subsequent mobilization of intracellular calcium.

Neurological, Psychiatric and Pain Disorders

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibuloocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders. Because of their physiological and pathophysiological significance, there is a need for new potent mGluR agonists and antagonists that display a high selectivity for mGluR subtypes, particularly the Group I receptor subtype, most particularly the mGluR5 subtype.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “G.I. reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind G.I. reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K, Holloway, R. H, Penagini, R., Blackshaw, L. A., Dent, J., 1995; Transient lower esophageal sphincter relaxation.

Gastroenterology 109,

pp. 601-610.

The wording “G.I. reflux” is herein defined as fluid from the stomach being able to pass into the esophagus, since the mechanical barrier is temporarily lost at such times. The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

Because of their physiological and pathophysiological significance, there is a continued need for new potent mGluR agonists and antagonists that display a high selectivity for mGluR subtypes, particularly the Group I receptor subtype. The object of the present invention is to provide compounds exhibiting an activity at metabotropic glutamate receptors (mGluRs), especially at the mGluR5 receptor.

### SUMMARY OF THE INVENTION

In one aspect of the invention, there is provided a compound according to formula I,
or a pharmaceutically acceptable salt or hydrate thereof: 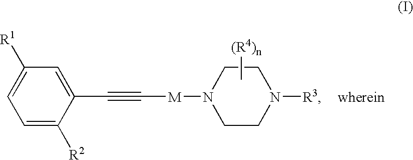
R¹ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S.

R² is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₀₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S.

R³ is selected from the group consisting of H, C(O)OC₁₋₆alkylhalo, C(O)OC₁₋₆alkyl, C(O)OC₂₋₆alkenyl, C(O)OC₂₋₆alkynyl, C(O)OC₀₋₆alkylC₃₋₆cycloalkyl, C(O)OC₀₋₆alkylaryl, C(O)OC₁₋₆alkylOR⁵, C(O)OC₁₋₆alkyl(CO)R⁵, C(O)OC₁₋₆alkylCO₂R⁵, C(O)OC₁₋₆alkylcyano, C(O)OC₀₋₆alkylNR⁵R⁶, C(O)OC₁₋₆alkyl(CO)NR⁵R⁶, C(O)OC₂₋₆alkylNR⁵(CO)R⁶, C(O)C₁₋₆alkylNR⁵(CO)NR⁵R⁶, C(O)OC₂₋₆alkylSR⁵, C(O)OC₁₋₆alkyl(SO)R⁵, C(O)OC₁₋₆alkylSO₂R⁵, C(O)OC₁₋₆alkyl(SO₂)NR⁵R⁶, C(O)OC₁₋₆alkylNR⁵(SO₂)R⁶, C(O)OC₂₋₆alkylNR⁵(SO₂)NR⁵R⁶, (CO)NR⁵R⁶, C(O)OC₁₋₆alkylNR⁵(CO)OR⁶, C(S)OC₁₋₆alkylhalo, C(S)OC₁₋₆alkyl, C(S)OC₂₋₆alkenyl, C(S)OC₂₋₆alkynyl, C(S)OC₀₋₆alkylC₃₋₆cycloalkyl, C(S)OC₀₋₆alkylaryl, C(S)OC₁₋₆alkylOR⁵, C(S)OC₁₋₆alkyl(CO)R⁵, C(S)OC₁₋₆alkylCO₂R⁵, C(S)OC₁₋₆alkylcyano, C(S)OC₀₋₆alkylNR⁵R⁶, C(S)OC₁₋₆alkyl(CO)NR⁵R⁶, C(S)OC₂₋₆alkylNR⁵(CO)R⁶, C(S)C₁₋₆alkylNR⁵(CO)NR⁵R⁶, C(S)OC₂₋₆alkylSR⁵, C(S)OC₁₋₆alkyl(SO)R⁵, C(S)OC₁₋₆alkylSO₂R⁵, C(S)OC₁₋₆alkyl(SO₂)NR⁵R⁶, C(S)OC₁₋₆alkylNR⁵(SO₂)R⁶, C(S)OC₂₋₆alkylNR⁵(SO₂)NR⁵R⁶, (CO)NR⁵R⁶, C(S)OC₁₋₆alkylNR⁵(CO)OR⁶, and a 5- or 6-membered ring containing one or more atoms independently selected from the group consisting of C, N, O and S;

R⁴ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₁₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, NR⁵, ═NOR⁵, ═O, ═S, SO₃R⁵, SO₃R⁵ and a 5- or 6-membered ring containing atoms independently selected from the group consisting of C, N, O and S.

M is selected from the group consisting of ═O, (CR⁵R⁶)ₘ and (CR⁵R⁶)ₘC(O).

R⁵ and R⁶ are independently selected from the group consisting of hydrogen, C₁₋₆alkyl, OC₁₋₆alkyl, C₃₋₇cycloalkyl, OC₃₋₇cycloalkyl, C₁₋₆alkylaryl, OC₁₋₆alkylaryl, aryl, and heteroaryl.

Any C₁₋₆alkyl, aryl or heteroaryl defined under R¹, R², R³, R⁴, R⁵ and R⁶ may be substituted by one or more A, where A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S.

Variable m is 0, 1, 2, or 3, while n is an integer between 0 and 8, inclusive.

In a further aspect of the invention there is provided pharmaceutical compositions comprising a therapeutically effective amount of a compound of formula I and a pharmaceutically acceptable diluent, excipient and/or inert carrier.

In yet a further aspect of the invention there is provided a pharmaceutical composition comprising a compound of formula I for use in the treatment of mGluR 5 receptor mediated disorders, and for use in the treatment of neurological disorders, psychiatric disorders, gastrointestinal disorders and pain disorders.

In still a further aspect of the invention there is provided the compound of formula I for use in therapy, especially for the treatment of mGluR 5 receptor mediated disorders, and for the treatment of neurological disorders, psychiatric disorders, gastrointestinal disorders and pain disorders.

A further aspect of the invention is the use of a compound according to formula I for the manufacture of a medicament for the treatment or prevention of obesity and obesity related conditions, as well as treating eating disorders by inhibition of excessive food intake and the resulting obesity and complications associated therewith.

In another aspect of the invention there is provided processes for the preparation of compounds of formula I and the intermediates used in the preparation thereof.

These and other aspects of the present invention are described in greater detail herein below.

### DETAILED DESCRIPTION OF THE PREFERRED EMBODIMENTS

The object of the present invention is to provide compounds exhibiting an activity at metabotropic glutamate receptors (mGluRs), especially at the mGluR 5 receptors. Listed below are definitions of various terms used in the specification and claims to describe the present invention.

For the avoidance of doubt it is to be understood that where in this specification a group is qualified by ‘hereinbefore defined’, ‘defined hereinbefore’ or ‘defined above’ said group encompasses the first occurring and broadest definition as well as each and all of the other definitions for that group.

For the avoidance of doubt it is to be understood that in this specification ‘C₁₋₆’ means a carbon group having 1, 2, 3, 4, 5 or 6 carbon atoms. Similarly ‘C₁₋₃’ means a carbon group having 1, 2, or 3 carbon atoms

In the case where a subscript is the integer 0 (zero) the group to which the subscript refers indicates that the group is absent.

In this specification, unless stated otherwise, the term “alkyl” includes both straight and branched chain alkyl groups and may be, but are not limited to methyl, ethyl, n-propyl, i-propyl, n-butyl, i-butyl, s-butyl, t-butyl, n-pentyl, i-pentyl, t-pentyl, neo-pentyl, n-hexyl or i-hexyl, t-hexyl. The term C₁₋₃alkyl has 1 to 3 carbon atoms and may be methyl, ethyl, n-propyl or i-propyl.

In this specification, unless stated otherwise, the term “cycloalkyl” refers to an optionally substituted, saturated cyclic hydrocarbon ring system. The term “C₃₋₇cycloalkyl” may be cyclopropyl, cyclobutyl, cyclopentyl, cyclohexyl or cycloheptyl. In this specification, unless stated otherwise, the term “alkoxy” includes both straight or branched alkoxy groups. C₁₋₃alkoxy may be, but is not limited to methoxy, ethoxy, n-propoxy or i-propoxy.

In this specification, unless stated otherwise, the term “bond” may be a saturated or unsaturated bond.

In this specification, unless stated otherwise, the term “halo” and “halogen” may be fluoro, chloro, bromo or iodo.

In this specification, unless stated otherwise, the term “alkylhalo” means an alkyl group as defined above, which is substituted with halo as described above. The term “C₁₋₆alkylhalo” may include, but is not limited to fluoromethyl, difluoromethyl, trifluoromethyl, fluoroethyl, difluoroethyl or bromopropyl. The term “OC₁₋₆alkylhalo” may include, but is not limited to fluoromethoxy, difluoromethoxy, trifluoromethoxy, fluoroethoxy or difluoroethoxy.

In this specification, unless stated otherwise, the term “alkenyl” includes both straight and branched chain alkenyl groups. The term “C₂₋₆alkenyl” refers to an alkenyl group having 2 to 6 carbon atoms and one or two double bonds, and may be, but is not limited to vinyl, allyl, propenyl, i-propenyl, butenyl, i-butenyl, crotyl, pentenyl, i-pentenyl and hexenyl.

In this specification, unless stated otherwise, the term “alkynyl” includes both straight and branched chain alkynyl groups. The term C₂₋₆alkynyl having 2 to 6 carbon atoms and one or two triple bonds, and may be, but is not limited to ethynyl, propargyl, butynyl, i-butynyl, pentynyl, i-pentynyl and hexynyl.

In this specification unless otherwise stated the term “aryl” refers to an optionally substituted monocyclic or bicyclic hydrocarbon ring system containing at least one unsaturated aromatic ring. Examples and suitable values of the term “aryl” are phenyl, naphthyl, 1,2,3,4-tetrahydronaphthyl, indyl and indenyl.

In this specification, unless stated otherwise, the term “heteroaryl” refers to an optionally substituted monocyclic or bicyclic unsaturated, ring system containing at least one heteroatom selected independently from N, O or S. Examples of “heteroaryl” may be, but are not limited to thiophene, thienyl, pyridyl, thiazolyl, furyl, pyrrolyl, triazolyl, imidazolyl, oxadiazolyl, oxazolyl, isoxazolyl, pyrazolyl, imidazolonyl, oxazolonyl, thiazolonyl, tetrazolyl and thiadiazolyl, benzoimidazolyl, benzooxazolyl, tetrahydrotriazolopyridyl, tetrahydrotriazolopyrimidinyl, benzofuryl, indolyl, isoindolyl, pyridonyl, pyridazinyl, pyrimidinyl, imidazopyridyl, oxazolopyridyl, thiazolopyridyl, pyridyl, imidazopyridazinyl, oxazolopyridazinyl, thiazolopyridazinyl and purinyl.

In this specification, unless stated otherwise, the term “alkylaryl”, “alkylheteroaryl” and “alkylcycloalkyl” refer to a substituent that is attached via the alkyl group to an aryl, heteroaryl and cycloalkyl group.

In this specification, unless stated otherwise, the term “heterocycloalkyl” refers to an optionally substituted, saturated cyclic hydrocarbon ring system wherein one or more of the carbon atoms are replaced with heteroatom. The term “heterocycloalkyl” includes but is not limited to pyrrolidine, tetrahydrofuran, tetrahydrothiophene, piperidine, piperazine, morpholine, thiomorpholine, tetrahydropyran, tetrahydrothiopyran.

In this specification, unless stated otherwise the term “5- or 6-membered ring containing atoms independently selected from C, N, O or S”, includes aromatic and heteroaromatic rings as well as carbocyclic and heterocyclic rings, which may be saturated, partially saturated or unsaturated. Examples of such rings may be, but are not limited to furyl, isoxazolyl, isothiazolyl, oxazolyl, pyrazinyl, pyrazolyl, pyridazinyl, pyridyl, pyrimidyl, pyrrolyl, thiazolyl, thienyl, imidazolyl, imidazolidinyl, imidazolinyl, triazolyl, morpholinyl, piperazinyl, piperidyl, piperidonyl, pyrazolidinyl, pyrazolinyl, pyrrolidinyl, pyrrolinyl, tetrahydropyranyl, thiomorpholinyl, phenyl, cyclohexyl, cyclopentyl and cyclohexenyl.

In this specification, unless stated otherwise, the term “═NR⁵” and “═NOR⁵” include imino- and oximo-groups carrying an R⁵ substituent and may be, or be part of, groups including, but not limited to iminoalkyl, iminohydroxy, iminoalkoxy, amidine, hydroxyamidine and alkoxyamidine.

In the case where a subscript is the integer 0 (zero) the group to which the subscript refers, indicates that the group is absent, i.e. there is a direct bond between the groups. In this specification unless stated otherwise the term “fused rings” refers to two rings which share 2 common atoms.

In this specification, unless stated otherwise, the term “bridge” means a molecular fragment, containing one or more atoms, or a bond, which connects two remote atoms in a ring, thus forming either bi- or tricyclic systems.

One embodiment of the invention relates to compounds of Formula I: 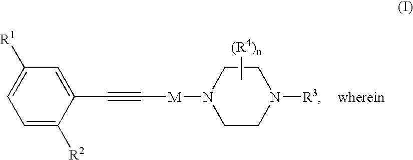
R¹ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S.

R² is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S.

R³ is selected from the group consisting of H, C(O)OC₁₋₆alkylhalo, C(O)OC₁₋₆alkyl, C(O)OC₂₋₆alkenyl, C(O)OC₂₋₆alkynyl, C(O)OC₀₋₆alkylC₃₋₆cycloalkyl, C(O)OC₀₋₆alkylaryl, C(O)OC₁₋₆alkylOR⁵, C(O)OC₁₋₆alkyl(CO)R⁵, C(O)OC₁₋₆alkylCO₂R⁵, C(O)OC₁₋₆alkylcyano, C(O)OC₀₋₆alkylNR⁵R⁶, C(O)OC₁₋₆alkyl(CO)NR⁵R⁶, C(O)OC₂₋₆alkylNR⁵(CO)R⁶, C(O)C₁₋₆alkylNR⁵(CO)NR⁵R⁶, C(O)OC₂₋₆alkylSR⁵, C(O)OC₁₋₆alkyl(SO)R⁵, C(O)OC₁₋₆alkylSO₂R⁵, C(O)OC₁₋₆alkyl(SO₂)NR⁵R⁶, C(O)OC₁₋₆alkylNR⁵(SO₂)R⁶, C(O)OC₂₋₆alkylNR⁵(SO₂)NR⁵R⁶, (CO)NR⁵R⁶, C(O)OC₁₋₆alkylNR⁵(CO)OR⁶, C(S)OC₁₋₆alkylhalo, C(S)OC₁₋₆alkyl, C(S)OC₂₋₆alkenyl, C(S)OC₂₋₆alkynyl, C(S)OC₀₋₆alkylC₃₋₆cycloalkyl, C(S)OC₀₋₆alkylaryl, C(S)OC₁₋₆alkylOR⁵, C(S)OC₁₋₆alkyl(CO)R⁵, C(S)OC₁₋₆alkylCO₂R⁵, C(S)OC₁₋₆alkylcyano, C(S)OC₀₋₆alkylNR⁵R⁶, C(S)OC₁₋₆alkyl(CO)NR⁵R⁶, C(S)OC₂₋₆alkylNR⁵(CO)R⁶, C(S)C₁₋₆alkylNR⁵(CO)NR⁵R⁶, C(S)OC₂₋₆alkylSR⁵, C(S)OC₁₋₆alkyl(SO)R⁵, C(S)OC₁₋₆alkylSO₂R⁵, C(S)OC₁₋₆alkyl(SO₂)NR⁵R⁶, C(S)OC₁₋₆alkylNR⁵(SO₂)R⁶, C(S)OC₂₋₆alkylNR⁵(SO₂)NR⁵R⁶, (CO)NR⁵R⁶, C(S)OC₁₋₆alkylNR⁵(CO)OR⁶, and a 5- or 6-membered ring containing one or more atoms independently selected from the group consisting of C, N, O and S;

R⁴ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, NR⁵, ═NOR⁵, ═O, ═S, SO₃R⁵, SO₃R⁵ and a 5- or 6-membered ring containing atoms independently selected from the group consisting of C, N, O and S.

M is selected from the group consisting of ═O, (CR⁵R⁶)ₘ and (CR⁵R⁶)ₘC(O).

R⁵ and R⁶ are independently selected from the group consisting of hydrogen, C₁₋₆alkyl, OC₁₋₆alkyl, C₃₋₇cycloalkyl, OC₃₋₇cycloalkyl, C₁₋₆alkylaryl, OC₁₋₆alkylaryl, aryl, and heteroaryl.

Any C₁₋₆alkyl, aryl or heteroaryl defined under R¹, R², R³, R⁴, R⁵ and R⁶ may be substituted by one or more A, where A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S.

Variable m is 0, 1, 2, or 3, while n is an integer between 0 and 8, inclusive.

A preferred subset of compounds of formula I are those in which n is 0. In this context, R³ preferably is selected from the group consisting of C(O)OC₁₋₆alkylhalo, C(O)OC₁₋₆alkyl, C(O)OC₂₋₆alkenyl, C(O)OC₂₋₆alkynyl, C(O)OC₀₋₆alkylC₃₋₆cycloalkyl, C(O)OC₀₋₆alkylaryl, C(O)OC₁₋₆alkylOR⁵, C(O)OC₁₋₆alkyl(CO)R⁵, C(O)OC₁₋₆alkylCO₂R⁵, C(O)OC₁₋₆alkylcyano, C(O)OC₀₋₆alkylNR⁵R⁶, C(O)OC₁₋₆alkyl(CO)NR⁵R⁶, C(O)OC₂₋₆alkylNR⁵(CO)R⁶, C(O)C₁₋₆alkylNR⁵(CO)NR⁵R⁶, C(O)OC₂₋₆alkylSR⁵, C(O)OC₁₋₆alkyl(SO)R⁵, C(O)OC₁₋₆alkylSO₂R⁵, C(O)OC₁₋₆alkyl(SO₂)NR⁵R⁶, C(O)OC₁₋₆alkylNR⁵(SO₂)R⁶, C(O)OC₂₋₆alkylNR⁵(SO₂)NR⁵R⁶, (CO)NR⁵R⁶, C(O)OC₁₋₆alkylNR⁵(CO)OR⁶, and a 5- or 6-membered ring containing one or more atoms independently selected from the group consisting of C, N, O and S. More preferably, R³ is C(O)OC₁₋₆alkyl, C(O)OC₀₋₆alkylaryl, C(O)OC₁₋₆alkylOR⁵, and (CO)NR⁵R⁶.

In other embodiments of the invention, R² is hydrogen or fluoro. Preferably, M is CR⁵R⁶. In this regard, R⁶ is preferably H, while R⁵ is preferably hydrogen, C₁₋₆alkyl, C₃₋₇cycloalkyl, C₁₋₆alkylaryl, aryl, or heteroaryl. In some embodiments, R⁵ is C₁₋₆alkylaryl. In other embodiments, R is C₃₋₇cycloalkyl. In yet other embodiments, R⁵ is heteroaryl. Preferred heteroaryl groups in this context include but are not limited to 2-, 3-, and 4-pyridyl; 2- and 3-thienyl; and 2- and 3-furanyl. In still other embodiments, R⁶ is aryl, phenyl being the most preferred.

Other embodiments of the invention relate to the following exemplary compounds of formula I:
4-[3-(3-Chloro-phenyl)-prop-2-ynyl]-piperazine-1-carboxylic acid ethyl ester,
4-(3-Phenyl-prop-2-ynyl)-piperazine-1-carboxylic acid ethyl ester,
4-[3-(3-Cyano-phenyl)-prop-2-ynyl]-piperazine-1-carboxylic acid ethyl ester,
4-(3-m-Tolyl-prop-2-ynyl)-piperazine-1-carboxylic acid ethyl ester,
4-[3-(3-Methoxy-phenyl)-prop-2-ynyl]-piperazine-1-carboxylic acid ethyl ester,
4-[3-(5-Cyano-2-fluoro-phenyl)-prop-2-ynyl]-piperazine-1-carboxylic acid ethyl ester,
4-[3-(2-Fluoro-5-methyl-phenyl)-prop-2-ynyl]-piperazine-1-carboxylic acid ethyl ester,
4-[3-(5-Chloro-2-fluoro-phenyl)-prop-2-ynyl]-piperazine-1-carboxylic acid ethyl ester,
4-[3-(3-Chloro-phenyl)-1-methyl-prop-2-ynyl]-piperazine-1-carboxylic acid ethyl ester,
4-[3-(3-Chloro-phenyl)-1-ethyl-prop-2-ynyl]-piperazine-1-carboxylic acid ethyl ester,
4-[3-(3-Chloro-phenyl)-1-isopropyl-prop-2-ynyl]-piperazine-1-carboxylic acid ethyl ester,
4-[1-tert-Butyl-3-(3-chloro-phenyl)-prop-2-ynyl]-piperazine-1-carboxylic acid ethyl ester,
4-[3-(3-Chloro-phenyl)-1-phenyl-prop-2-ynyl]-piperazine-1-carboxylic acid ethyl ester,
4-[1-(3-Chloro-phenylethynyl)-butyl]-piperazine-1-carboxylic acid ethyl ester,
4-[1-(3-Chloro-phenylethynyl)-3-methyl-butyl]-piperazine-1-carboxylic acid ethyl ester,
4-[1-Benzyloxymethyl-3-(3-chloro-phenyl)-prop-2-ynyl]-piperazine-1-carboxylic acid ethyl ester,
4-[3-(3-Chloro-phenyl)-1-cyclopropyl-prop-2-ynyl]-piperazine-1-carboxylic acid ethyl ester,
4-[1-(3-Chloro-phenylethynyl)-pentyl]-piperazine-1-carboxylic acid ethyl ester,
4-[3-(3-Chloro-phenyl)-1-thiophen-2-yl-prop-2-ynyl]-piperazine-1-carboxylic acid ethyl ester,
4-[3-(3-Chloro-phenyl)-1-thiophen-3-yl-prop-2-ynyl]-piperazine-1-carboxylic acid ethyl ester,
4-[3-(3-Chloro-phenyl)-1-furan-2-yl-prop-2-ynyl]-piperazine-1-carboxylic acid ethyl ester,
4-[3-(3-Chloro-phenyl)-1-ethyl-prop-2-ynyl]-piperazine-1-carboxylic acid tert-butyl ester,
1-[3-(3-Chloro-phenyl)-1-ethyl-prop-2-ynyl]-piperazine,
4-[3-(3-Chloro-phenyl)-1-ethyl-prop-2-ynyl]-piperazine-1-carboxylic acid isopropyl ester,
4-[3-(3-Chloro-phenyl)-1-ethyl-prop-2-ynyl]-piperazine-1-carboxylic acid propyl ester,
4-[3-(3-Chloro-phenyl)-1-ethyl-prop-2-ynyl]-piperazine-1-carboxylic acid isobutyl ester,
4-[3-(3-Chloro-phenyl)-1-ethyl-prop-2-ynyl]-piperazine-1-carboxylic acid butyl ester,
4-[3-(3-Chloro-phenyl)-1-ethyl-prop-2-ynyl]-piperazine-1-carboxylic acid 2,2-dimethyl-propyl ester,
4-[3-(3-Chloro-phenyl)-1-ethyl-prop-2-ynyl]-piperazine-1-carboxylic acid pentyl ester,
4-[3-(3-Chloro-phenyl)-1-ethyl-prop-2-ynyl]-piperazine-1-carboxylic acid 2-methoxy-ethyl ester,
4-[3-(3-Chloro-phenyl)-1-ethyl-prop-2-ynyl]-piperazine-1-carboxylic acid phenyl ester,
4-[3-(3-Chloro-phenyl)-1-ethyl-prop-2-ynyl]-piperazine-1-carboxylic acid benzyl ester,
4-[3-(3-Chloro-phenyl)-1-pyridin-3-yl-prop-2-ynyl]-piperazine-1-carboxylic acid ethyl ester,
4-[3-(3-Chloro-phenyl)-1-(2,4-difluoro-phenyl)-prop-2-ynyl]-piperazine-1-carboxylic acid ethyl ester,
4-[3-(3-Chloro-phenyl)-1-(2-methoxy-phenyl)-prop-2-ynyl]-piperazine-1-carboxylic acid ethyl ester,
4-[3-(3-Chloro-phenyl)-1-(2-chloro-phenyl)-prop-2-ynyl]-piperazine-1-carboxylic acid ethyl ester,
4-[3-(3-Chloro-phenyl)-1-o-tolyl-prop-2-ynyl]-piperazine-1-carboxylic acid ethyl ester,
4-[3-(3-Chloro-phenyl)-1-m-tolyl-prop-2-ynyl]-piperazine-1-carboxylic acid ethyl ester,
4-[3-(3-Chloro-phenyl)-1-(6-methoxy-pyridin-3-yl)-prop-2-ynyl]-piperazine-1-carboxylic acid ethyl ester, and
4-[3-(3-Chloro-phenyl)-1-(2-chloro-pyridin-3-yl)-prop-2-ynyl]-piperazine-1-carboxylic acid ethyl ester,
Ethyl 4-[3-(5-chloro-2-fluorophenyl)-1-ethylprop-2-yn-1-yl]piperazine-1-carboxylate,
Ethyl 4-[3-(3-chlorophenyl)-1-(5-methyl-2-furyl)prop-2-yn-1-yl]piperazine-1-carboxylate,
Ethyl 4-{3-(3-chlorophenyl)-1-[5-(methoxycarbonyl)-2-furyl]prop-2-yn-1-yl}piperazine-1-carboxylate,
2,2,2-Trifluoroethyl 4-[3-(3-chlorophenyl)-1-(2-furyl)prop-2-yn-1-yl]piperazine-1-carboxylate,
Ethyl 4-{3-(3-chlorophenyl)-1-[5-(hydroxymethyl)-2-furyl]prop-2-yn-1-yl}piperazine-1-carboxylate,
Ethyl (3S)-4-[(1R)-3-(3-chlorophenyl)-1-(2-furyl)prop-2-yn-1-yl]-3-methylpiperazine-1-carboxylate,
Ethyl (3S)-4-[(1S)-3-(3-chlorophenyl)-1-(2-furyl)prop-2-yn-1-yl]-3-methylpiperazine-1-carboxylate,
Ethyl (3R)-4-[(1S)-3-(3-chlorophenyl)-1-ethylprop-2-yn-1-yl]-3-methylpiperazine-1-carboxylate,
Ethyl (3R)-4-[(1R)-3-(3-chlorophenyl)-1-(2-furyl)prop-2-yn-1-yl]-3-methylpiperazine-1-carboxylate,
Ethyl (3R)-4-[(1R)-3-(3-chlorophenyl)-1-ethylprop-2-yn-1-yl]-3-methylpiperazine-1-carboxylate,
Ethyl (3S)-4-[(1S)-3-(3-chlorophenyl)-1-ethylprop-2-yn-1-yl]-3-methylpiperazine-1-carboxylate,
Ethyl (3S)-4-[(1R)-3-(3-chlorophenyl)-1-methylprop-2-yn-1-yl]-3-methylpiperazine-1-carboxylate,
4-[3-(3-Chloro-phenyl)-prop-2-ynyl]-piperazine-1-carboxylic acid tert-butyl ester,
4-[1-(Tert-Butoxycarbonylamino-methyl)-3-(3-chloro-phenyl)-prop-2-ynyl]-piperazine-1-carboxylic acid ethyl ester,
4-[3-(3-Chloro-phenyl)-1-triisopropylsilyloxymethyl-prop-2-ynyl]-piperazine-1-carboxylic acid ethyl ester,
Ethyl 4-[3-(3-chlorophenyl)-1-(ethoxymethyl)prop-2-yn-1-yl]piperazine-1-carboxylate,
4-[1-Aminomethyl)-3-(3-chloro-phenyl)-prop-2-ynyl]-piperazine-1-carboxylic acid ethyl ester,
4-[3-(3-Chloro-phenyl)-1-hydroxymethyl-prop-2-ynyl]-piperazine-1-carboxylic acid ethyl ester,
4-[3-(3-Chloro-phenyl)-1-methoxymethyl-prop-2-ynyl]-piperazine-1-carboxylic acid ethyl ester,
4-(3-Phenyl-propynoyl)-piperazine-1-carboxylic acid ethyl ester
Ethyl 4-[3-(3-Chloro-phenyl)-1,1-dimethyl-prop-2-ynyl]-piperazine-1-carboxylic acid ethyl ester,
4-[3-(3-Chloro-phenyl)-1-ethyl-prop-2-ynyl]-piperazine-1-carboxylic acid methyl ester, and
4-[3-(3-Chloro-phenyl)-prop-2-ynyl]-piperazine-1-caroxylic acid 2-methoxy-ethyl ester.

Embodiments of the invention include salt forms of the compounds of Formula I. Salts for use in pharmaceutical compositions will be pharmaceutically acceptable salts, but other salts may be useful in the production of the compounds of Formula I. A suitable pharmaceutically acceptable salt of the compounds of the invention is, for example, an acid-addition salt, for example an inorganic or organic acid. In addition, a suitable pharmaceutically acceptable salt of the compounds of the invention is an alkali metal salt, an alkaline earth metal salt or a salt with an organic base.

Other pharmaceutically acceptable salts and methods of preparing these salts may be found in, for example, Remington's Pharmaceutical Sciences (18^{th} Edition, Mack Publishing Co.) 1990.

Some compounds of formula I may have chiral centres and/or geometric isomeric centres (E- and Z-isomers), and it is to be understood that the invention encompasses all such optical, diastereoisomeric and geometric isomers.

The invention also relates to any and all tautomeric forms of the compounds of Formula I.

The invention further relates to hydrate and solvate forms of the compounds of Formula I.

Pharmaceutical Composition

According to one aspect of the present invention there is provided a pharmaceutical composition comprising as active ingredient a therapeutically effective amount of the compound of Formula I, or salts, solvates or solvated salts thereof, in association with one or more pharmaceutically acceptable diluent, excipients and/or inert carrier.

The composition may be in a form suitable for oral administration, for example as a tablet, pill, syrup, powder, granule or capsule, for parenteral injection (including intravenous, subcutaneous, intramuscular, intravascular or infusion) as a sterile solution, suspension or emulsion, for topical administration e.g. as an ointment, patch or cream or for rectal administration e.g. as a suppository.

In general the above compositions may be prepared in a conventional manner using one or more conventional excipients, pharmaceutical acceptable diluents and/or inert carriers.

Suitable daily doses of the compounds of formula I in the treatment of a mammal, including man are approximately 0.01 to 250 mg/kg bodyweight at peroral administration and about 0.001 to 250 mg/kg bodyweight at parenteral administration. The typical daily dose of the active ingredients varies within a wide range and will depend on various factors such as the relevant indication, severity of the illness being treated, the route of administration, the age, weight and sex of the patient and the particular compound being used, and may be determined by a physician.

Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR 5 and for inhibiting neuronal damage caused by excitatory activation of mGluR 5. The compounds may be used to produce an inhibitory effect of mGluR 5 in mammals, including man.

The mGluR Group I receptor including mGluR 5 are highly expressed in the central and peripheral nervous system and in other tissues. Thus, it is expected that the compounds of the invention are well suited for the treatment of mGluR 5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR 5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined hereinbefore, for use in treatment of pain related to migraine, inflammatory pain, neuropathic pain disorders such as diabetic neuropathies, arthritis and rheumatoid diseases, low back pain, post-operative pain and pain associated with various conditions including angina, renal or biliary colic, menstruation, migraine and gout.

The invention relates to compounds of Formula I as defined hereinbefore, for use in treatment of stroke, head trauma, anoxic and ischemic injuries, hypoglycemia, cardiovascular diseases and epilepsy.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a compound according to Formula I, for the manufacture of a medicament for the inhibition of transient lower esophageal sphincter relaxations, for the treatment of GERD, for the prevention of G.I. reflux, for the treatment regurgitation, treatment of asthma, treatment of laryngitis, treatment of lung disease and for the management of failure to thrive. A further embodiment of the invention relates to the use of a compound according to formula I for the manufacture of a medicament for the treatment or prevention of functional gastrointestinal disorders, such as functional dyspepsia (FD). Yet another aspect of the invention is the use of a compound according to formula I for the manufacture of a medicament for the treatment or prevention of irritable bowel syndrome (IBS), such as constipation predominant IBS, diarrhea predominant IBS or alternating bowel movement predominant IBS.

A further aspect of the invention is the use of a compound according to formula X for the manufacture of a medicament for the treatment or prevention of obesity and obesity related conditions, as well as treating eating disorders by inhibition of excessive food intake and the resulting obesity and complications associated therewith.

The invention also provides a method of treatment of mGluR 5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand. The term “disorder”, unless stated otherwise, means any condition and disease associated with metabotropic glutamate receptor activity.

Non-Medical Use

In addition to their use in therapeutic medicine, the compounds of Formula I, salts or hydrates thereof, are also useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

Methods of Preparation

Another aspect of the present invention provides processes for preparing compounds of Formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It is also to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994). Techniques for purification of intermediates and final products include for example, straight and reversed phase chromatography on column or rotating plate, recrystallization, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

The term “reflux” shall mean, unless otherwise stated, in reference to an employed solvent a temperature at or above the boiling point of named solvent.

Abbreviations

aq. Aqueous
atm atmosphere
BINAP 2,2′Bis(diphenylphosphino)-1,1′-binaphthyl
Boc, BOC tert-butoxycarbonyl
CDI N,N′-Carbonyldiimidazole
dba Dibenzylideneacetone
DCC N,N-Dicyclohexylcarbodiimide
DCM Dichloromethane
DEA N,N-Diisopropylethylamine
DIBAL-H Diisobutylaluminum hydride
DIC N,N′-Diisopropylcarbodiimide
DMAP N,N-Dimethyl-4-aminopyridine
DMF Dimethylformamide
DMSO Dimethylsulfoxide
DPPF 1,1′-Bis(diphenylphosphino)ferrocene
EA or EtOAc Ethyl acetate
EDC, EDCl N-[3-(dimethylamino)propyl]-N′-ethylcarbodiimide hydrochloride
Et Ethyl
Et₂O Diethyl ether
EtI Iodoethane
EtOH Ethanol
Et₃N Triethylamine
Fmoc, FMOC 9-Fluorenylmethoxycarbonyl
h hour(s)
HBTU O-(Benzotriazol-1-yl)-N,N,N′,N′-tetramethyluronium hexafluorophosphate
HetAr Heteroaryl
HOBt N-Hydroxybenzotriazole
HPLC, LC high performance liquid chromatography
LCMS HPLC mass spec
MCPBA m-chlorbenzoic acid
Me Methyl
MeCN Acetonitrile
MeI Iodomethane
MeMgCl methyl magnesium chloride
MeOH Methanol
min Minutes
MS mass spec
NaOAc sodium acetate
nBu normal butyl
nBuLi, n-BuLi 1-butyllithium
NCS N-chlorosuccinimide
NMR nuclear magnetic resonance
o.n. over night
OAc acetate
OMs mesylate or methane sulfonate ester
OTs tosylate, toluene sulfonate or 4-methylbenzene sulfonate ester
PPTS pyridinium p-toluenesulfonate
pTsOH p-toluenesulfonic acid
RT, rt, r.t. room temperature
s seconds
sat. Saturated
SPE solid phase extraction
TBAF tetrabutylammonium fluoride
tBu, t-Bu tert-butyl
tBuOH, t-BuOH tert-butanol
TEA Triethylamine
TFA trifluoroacetic acid
THF Tetrahydrofuran
TMS tetramethylsilane

Compounds of Formula A wherein R³ and R⁴ are defined as in Formula I can be prepared as shown in Scheme 1. The piperazine intermediate II can be first N-alkylated with propargyl halides to give intermediate III followed by Sonogashira coupling (see Miki, Y., Momotake, A., Arai, T.: Org. Biomol. Chem., 2003, 1, 2655-2660) with various aryl halides to afford product A.

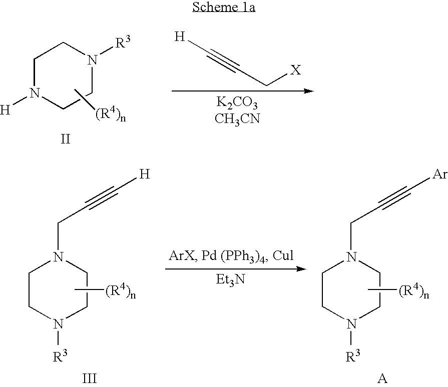

This reaction may also be accomplished in a single-pot by combining the amine, aryl iodide, and acetylene (using a small amount of DCM to help solubilize for solid piperazines) and heating at temperatures such as 60-100° C. in the presence of the required palladium and copper catalysts. The piperazine may itself act as the amine base, negating the need for an additional base such as triethylamine.

Alternatively, compounds of formula A can be prepared by reaction of amines of formula II with a suitable propargyl halide of formula IV (Scheme 1b). The propargyl halide intermediates IV (X═Cl, Br or I) can be prepared from the corresponding propargyl alcohol derivative utilizing processes established in the art (e.g. PBr₃, CBr₄, NBS, NCS). The various propargyl alcohol derivatives can in turn be obtained from a Sonogashira coupling between aromatic halides and prop-2-yn-1-ol.

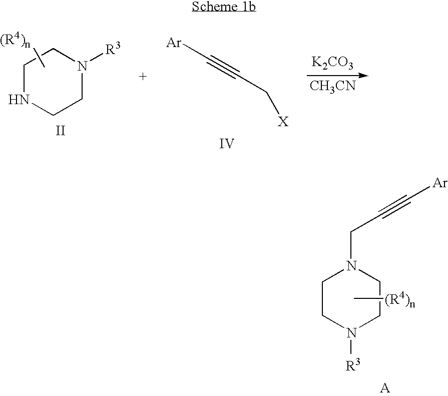

Compounds of Formula B wherein R³, R⁴ and R⁵ are defined as in Formula I can be prepared using the recently published three-component coupling of aldehydes, alkynes and piperazines (amines) in water under catalytic conditions (Scheme 2a). The catalysts that may effect the coupling include, for example, AuBr₃, AuCl, AuI, AgI, and AgBr (see Wei, C. Li, C-J.: J. Am. Chem. Soc. 2003, 125, 9584-9585; Wei, C., Zigang, L., Li, C-J.: Org. Lett 2003, 5, 4473-4475). 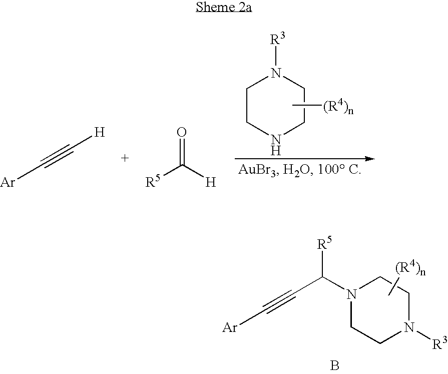
Scheme 2a can also be carried out in a microwave oven using copper salts; this has the benefit of being more cost effective than the approach using gold or silver salts. (see Shi, L.; Tu, Y.-Q.; Wang, M.; Zhang, F.-M.; Fan, C.-H. Organic Letters 2004, 6, 1001-1003).

Alternatively, compounds of Formula B wherein R³ is COOR can also be obtained from intermediates V, which may be derived using a suitably protected precursor, such as the Boc-protected piperazine, with assembly under the three component coupling conditions described above or by using displacement of a propargyl halide as in scheme 1 wherein R¹═H. The resulting piperazine intermediate V may be subsequently treated with a variety of chloroformates in the presence of a base in an appropriate solvent to afford the final compounds B (Scheme 2b).

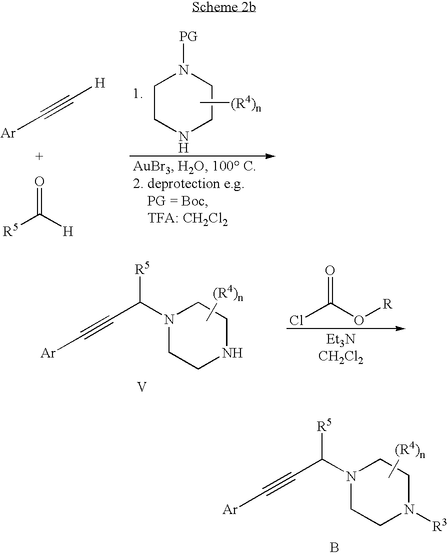

In the event that a masking group G is attached to the acetylene as shown in Scheme 3 below, the masked acetylene can be coupled to the piperazine derivative containing an appropriate R group to give the acetylene-masked intermediate VII. Subsequent removal of the G group followed by Sonogashira coupling with different aryl halides delivers compounds of general formula B.

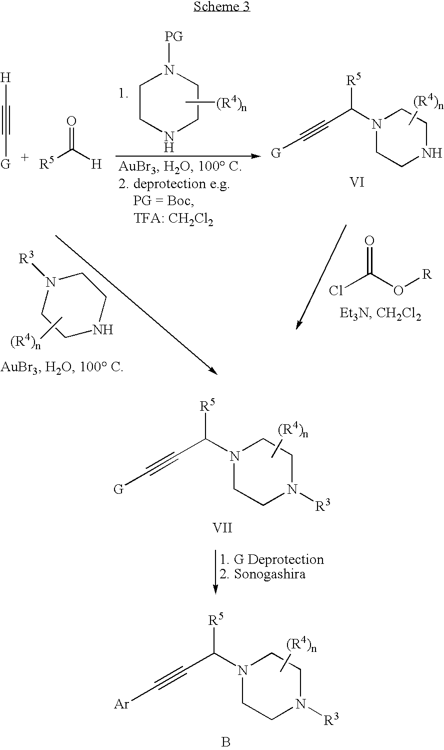

A variation on the synthetic approach to compounds B begins with the protected piperazine followed by immediate deprotection to give the versatile intermediate piperazine VI. Compounds of formula B wherein R³ is COOR can be formed by introduction of the COOR via the chloroformates to provide intermediate VII which can then be used to introduce various aryl groups by acetylene unmasking and subsequent Sonogashira coupling. In the approaches outlined in Scheme 3, G is a temporary masking group (e.g. triethylsilyl, triisopropylsilyl) that can be removed with tetrabutylammonium fluoride or K₂CO₃ in MeOH.

Compounds of Formula I wherein M=CO may be prepared by coupling an aryl propiolic acid with a suitable piperazine using a coupling reagent such as EDCI in the presence of catalyst such as DMAP, in a polar aprotic solvent such as DMF. 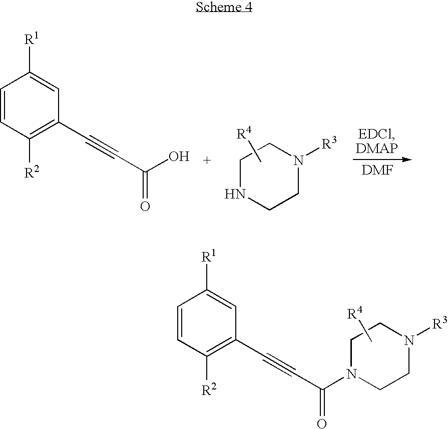
Compounds of Formula I wherein M=CMe₂ may be prepared by copper catalyzed alkylation of a tertiary propargylic chloride with the suitable piperazine to form the propargylic piperazine without rearrangement, (see Zaragoza, F.; Stephensen, H.; Knudsen, S. M.; Pridal, L.; Wulff, B. S.; Rimvall, K. J. Med. Chem. 2004, 47, 2833-2838) followed by coupling to an aryl bromide or iodide using a palladium catalyst such as bis(triphenylphosphine)palladium(II) chloride in the presence of a copper salt such as cuprous iodide and an amine base such as triethylamine.

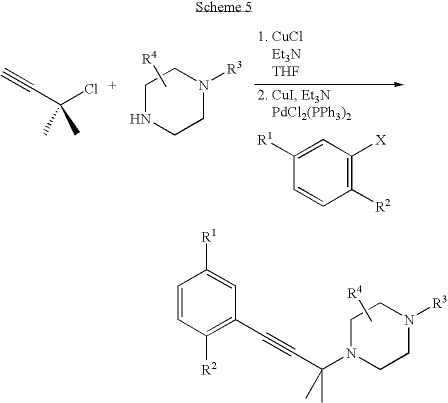

The invention will now be illustrated by the following non-limiting examples.

General Methods

All starting materials are commercially available or earlier described in the literature. The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.).

Preparative reversed phase chromatography was run on a Gilson autopreparative HPLC with a diode array detector using an XTerra MS C8, 19×300 mm, 7 mm as column.

Purification by a chromatotron was performed on rotating silica gel/gypsum (Merck, 60 PF-254 with calcium sulphate) coated glass sheets, with coating layer of 1, 2, or 4 mm using a TC Research 7924T chromatotron. Purification of products were also done by flash chromatography in silica-filled glass columns or SPE cartridges pre-filled with silica gel from Varian (Mega BE-SI 5G or 10G).

Microwave heating was performed in a Smith Synthesizer Single-mode microwave cavity producing continuous irradiation at 2450 MHz (Personal Chemistry AB, Uppsala, Sweden).

The following compounds were synthesized according to Scheme 1.

### EXAMPLE 1

### 4-Prop-2-ynyl-piperazine-1-carboxylic acid ethyl ester

To a stirred suspension of K₂CO₃ (11.6 g, 84.0 mmol) in acetonitrile cooled to 0° C. was added piperazine-1-carboxylic acid ethyl ester (31.0 ml, 210 mmol), followed by propargyl bromide (3.75 mL, 34 mmol). The reaction was allowed to stir for 1.5 hours. Reaction mixture was diluted with CH₂Cl₂, washed with water, then brine followed by drying over sodium sulphate (anhydrous). The crude organic product was concentrated in vacuo and purified by flash chromatography afforded quantitative yield of the product as a yellow oil. ¹H NMR (CDCl₃) δ (ppm): 4.14 (q, 2H), 3.51(t, 4H), 3.33 (d, 2H), 2.53 (t, 4H), 2.28 (t, 1H) 1.27 (t, 3H).

### EXAMPLE 2

### 4-[3-(3-Chloro-phenyl)-prop-2-ynyl]-piperazine-1-carboxylic acid ethyl ester

See Miki, Y., Momotake, A., Arai, T.: Org. Biomol. Chem., 2003, 1, 2655-2660. A mixture of 4-Prop-2-ynyl-piperazine-1-carboxylic acid ethyl ester (0.10 g, 0.55 mmol), metachloroiodobenzene (0.089 mL, 0.72 mmol), bis(triphenylphosphine)palladium (II) chloride (19 mg, 0.03 mmol) and copper iodide (11 mg, 0.06 mmol) in triethylamine (5 mL) was stirred at 40° C. for 19 h. Reaction mixture was poured into water and extracted with EtOAc. The organic layer was washed with saturated NH₄Cl solution followed by brine, then dried (Na₂SO₄), filtered and concentrated onto silica gel. Chromatography (SPE) using 1:1 EtOAc/CH₂Cl₂ as eluent. ¹H NMR showed triethylamine remaining. Crude product was triturated with hexanes 2× and concentrated under high vacuum following a second extraction (EtOAc and NH₄Cl). Re-Chromatography (SPE) eluting with 30% EtOAc/hexanes followed by 100% EtOAc to give 32 mg (19%) of the desired compound as a yellow oil. ¹H NMR (CDCl₃) δ (ppm): 7.43 (td, 1H), 7.25-7.33 (m, 3H), 4.16 (q, 2H), 3.55-3.58 (m, 6H), 2.60 (t, 4H), 1.28 (t, 3H).

### EXAMPLE 3

### 4-(3-Phenyl-prop-2-ynyl)-piperazine-1-carboxylic acid ethyl ester

A mixture of 4-Prop-2-ynyl-piperazine-1-carboxylic acid ethyl ester (0.10 g, 0.55 mmol), iodobenzene (0.064 mL, 0.57 mmol), bis(triphenylphosphine)palladium (II) chloride (15 mg, 0.02 mmol) and copper iodide (8 mg, 0.04 mmol) in triethylamine (5 mL) was stirred at 40° C. for 19 h. Reaction mixture was poured into water (20 mL) and extracted with EtOAc (50 mL). The organic layer was washed with saturated NH₄Cl solution (4×20 mL) followed by brine (20 mL), then dried (Na₂SO₄), filtered and concentrated onto silica gel. Chromatography (SPE) using 40-70% EtOAc/hexanes as eluent gave 75 mg (63%) of the desired compound as a yellow oil. ¹H NMR (CDCl₃) δ (ppm): 7.42-7.46 (m, 2H), 7.30-7.33 (m, 3H), 4.16 (q, 2H), 3.53-3.60 (m, 6H), 2.61 (t, 4H), 1.28 (t, 3H).

### EXAMPLE 4

### 4-[3-(3-Cyano-phenyl)-prop-2-ynyl]-piperazine-1-carboxylic acid ethyl ester

A mixture of 4-Prop-2-ynyl-piperazine-1-carboxylic acid ethyl ester (0.10 g, 0.55 mmol), 3-Iodo-benonitrile (0.16 g, 0.70 mmol), bis(triphenylphosphine)palladium (II) chloride (19 mg, 0.03 mmol) and copper iodide (11 mg, 0.06 mmol) in triethylamine (5 mL) was stirred at 40° C. for 19 h. Reaction mixture was poured into water (25 mL) and extracted with EtOAc (50 mL). The organic layer was washed with saturated NH₄Cl solution (4×15 mL) followed by brine (20 mL), then dried (Na₂SO₄), filtered and concentrated onto silica gel. Chromatography (SPE) eluting with 30-70-100% EtOAc/hexanes afforded 75 mg (46%) of the desired compound as a yellow oil. ¹H NMR (CDCl₃) δ (ppm): 7.70-7.73 (m, 1H), 7.65 (dt, 1H), 7.60 (dt, 1H), 7.44 (td, 1H), 4.16 (q, 2H), 3.51-3.60 (m, 6H), 2.60 (t, 4H), 1.28 (t, 3H).

### EXAMPLE 5

### 4-(3-m-Tolyl-prop-2-ynyl)-piperazine-1-carboxylic acid ethyl ester

A mixture of 4-Prop-2-ynyl-piperazine-1-carboxylic acid ethyl ester (0.10 g, 0.55 mmol), 1-Iodo-3-methylbenzene (0.150 mL, 1.17 mmol), bis(triphenylphosphine)palladium (II) chloride (19 mg, 0.03 mmol) and copper iodide (11 mg, 0.06 mmol) in triethylamine (5 mL) was stirred at 40° C. for 19 h. Reaction mixture was poured into water (25 mL) and extracted with EtOAc (50 mL). The organic layer was washed with saturated NH₄Cl solution (4×15 mL) followed by brine (20 mL), then dried (Na₂SO₄), filtered and concentrated onto silica gel. Chromatography (SPE) eluting with 30-100% EtOAc/hexanes afforded 97 mg (62%) of the desired compound as a yellow oil. ¹H NMR (CDCl₃) δ (ppm): 7.11-7.29 (m, 4H), 4.16 (q, 2H), 3.52-3.60 (m, 6H), 2.61 (t, 4H), 2.34 (s, 3H), 1.28 (t, 3H).

### EXAMPLE 6

### 4-[3-(3-Methoxy-phenyl)-prop-2-ynyl]-piperazine-1-carboxylic acid ethyl ester

A mixture of 4-Prop-2-ynyl-piperazine-1-carboxylic acid ethyl ester (0.10 g, 0.55 mmol), 1-Iodo-3-methoxy-benzene (0.100 mL, 0.84 mmol), bis(triphenylphosphine)palladium (II) chloride (19 mg, 0.03 mmol) and copper iodide (11 mg, 0.06 mmol) in triethylamine (5 mL) was stirred at 40° C. for 19 h. Reaction mixture was poured into water (25 mL) and extracted with EtOAc (50 mL). The organic layer was washed with saturated NH₄Cl solution (4×15 mL) followed by brine (20 mL), then dried (Na₂SO₄), filtered and concentrated onto silica gel. Chromatography (SPE) eluting with 30-100% EtOAc/hexanes afforded 84 mg (51%) of the desired compound as a yellow oil. ¹H NMR (CDCl₃) δ (ppm): 7.23 (t, J=8 Hz, 1H), 7.04 (dt, J=8, 1 Hz, 1H), 6.98 (dd, J=3, 2 Hz, 1H), 6.89 (ddd, J=8, 3, 1 Hz, 1H), 4.16 (q, J=7 Hz, 2H), 4.16 (q, J=7 Hz, 2H), 3.82 (s, 3H), 3.52-3.60 (m, 6H), 2.61 (t, J=5 Hz, 4H), 1.28 (t, J=7 Hz, 3H).

### EXAMPLE 7

### 4-[3-(5-Cyano-2-fluoro-phenyl)-prop-2-ynyl]-piperazine-1-carboxylic acid ethyl ester

See Hundertmark, T., Littke, A. F., Buchwald, S. L, Fu, G. C.: Org. Lett. 2000, 2, 12, 1729-1731. 4-Prop-2-ynyl-piperazine-1-carboxylic acid ethyl ester (0.22 g, 0.96 mmol), 3-bromo-4-fluorobenzonitrile (0.23 g, 1.2 mmol) and diisopropylamine (0.17 mL, 1.2 mmol) were dissolved in dioxane (1 mL), and the solution degassed with argon for ˜10 minutes. Bis(methylcyanate)palladium(II)chloride (12 mg, 0.05 mmol), copper iodide (4 mg, 0.02 mmol) and tri-tert-butylphosphine (0.014 mL, 0.06 mmol) were added, and the reaction was sealed and allowed to stir for 16 h. Reaction mixture was diluted with EtOAc (5 mL) and filtered over celite using EtOAc. The organic layer was washed with NH₄Cl solution (4×10 mL), then dried (Na₂SO₄), filtered and concentrated onto silica gel. Chromatography (SPE) eluting with 30-50% EtOAc/hexanes afforded 137 mg (45%) of the title compound as a yellow oil. ¹H NMR (CDCl₃) δ (ppm): 7.75 (dd, 1H), 7.2 (ddd, 1H), 7.21 (t, 1H), 4.16 (q, 2H), 3.62 (s, 2H), 3.57 (t, 4H), 2.61 (t, 4H), 1.28 (t, 3H).

### EXAMPLE 8

### 4-[3-(2-Fluoro-5-methyl-phenyl)-prop-2-ynyl]-piperazine-1-carboxylic acid ethyl ester

4-Prop-2-ynyl-piperazine-1-carboxylic acid ethyl ester (0.22 g, 0.96 mmol), 3-bromo-4-fluorotoluene (0.14 mL, 1.2 mmol) and diisopropylamine (0.17 mL, 1.2 mmol) were dissolved in dioxane (1 mL), and the solution degassed with argon for ˜10 minutes. Bis(methylcyanate)palladium(II)chloride (12 mg, 0.05 mmol), copper iodide (4 mg, 0.02 mmol) and tri-tert-butylphosphine (0.014 mL, 0.06 mmol) were added, and the reaction was sealed and allowed to stir for 16 h. Reaction mixture was diluted with EtOAc (5 mL) and filtered over celite using EtOAc. The organic layer was washed with NH₄Cl solution (4×10 mL), then dried (Na₂SO₄), filtered and concentrated onto silica gel. Chromatography (SPE) eluting with 30-50% EtOAc/hexanes afforded 44 mg (15%) of the title compound as a yellow oil. ¹H NMR (CDCl₃) δ (ppm): 7.23 (dd, 1H), 7.05-7.12 (m, 1H), 6.95 (t, 1H), 4.16 (q, 2H), 3.60 (s, 2H), 3.57 (t, 4H), 2.62 (t, 4H), 2.30 (s, 3H), 1.28 (t, 3H).

### EXAMPLE 9

### 4-[3-(5-Chloro-2-fluoro-phenyl)-prop-2-ynyl]-piperazine-1-carboxylic acid ethyl ester

4-Prop-2-ynyl-piperazine-1-carboxylic acid ethyl ester (0.22 g, 0.96 mmol), 2-bromo-1-chloro-1-fluorobenzene (0.14 mL, 1.2 mmol) and diisopropylamine (0.17 mL, 1.2 mmol) were dissolved in dioxane (1 mL), and the solution degassed with argon for ˜10 minutes. Bis(methylcyanate)palladium(II)chloride (12 mg, 0.05 mmol), copper iodide (4 mg, 0.02 mmol) and tri-tert-butylphosphine (0.014 mL, 0.06 mmol) were added, and the reaction was sealed and allowed to stir for 16 h. Reaction mixture was diluted with EtOAc (5 mL) and filtered over celite using EtOAc. The organic layer was washed with NHCl solution (4×10 mL), then dried (Na₂SO₄), filtered and concentrated onto silica gel. Chromatography (SPE) eluting with 30-50% EtOAc/hexanes afforded 113 mg (36%) of the title compound as a yellow oil. ¹H NMR (CDCl₃) δ (ppm): 7.41 (dd, 1H), 7.26 (ddd, 1H), 7.02 (t, 1H), 4.16 (q, 2H), 3.60 (s, 2H), 3.56 (t, 4H), 2.61 (t, 4H), 1.28 (t, 3H).

The following compounds were synthesized according to Scheme 2

### EXAMPLE 10

### 4-[3-(3-Chloro-phenyl)-1-methyl-prop-2-ynyl]-piperazine-1-carboxylic acid ethyl ester

Water (2.5 mL) was deoxygenated with argon for 10 minutes in a pressure flask. 3-chloro-1-ethynyl-benzene (1.0 g, 3.7 mmol), Ethyl-1-piperizinecarboxylate (0.4 mL, 2.7 mmol), gold (III) bromide (catalytic) and acetaldehyde (0.14 mL, 2.4 mmol) were added, and the reaction heated to 100° C., sealed and stirred for 16 h. Reaction mixture was extracted with EtOAc and washed with brine, then dried (Na₂SO₄), filtered and concentrated onto silica gel. Chromatography (silica gel ˜30 g) eluting with 30% EtOAc/hexanes afforded 51 mg (6.6%) of the title compound as a brown oil. ¹H NMR (CDCl₃) δ (ppm): 7.42 (m, 1H), 7.21=7.34 (m, 3H), 4.16 (q, 2H), 3.74 (q, 1H), 3.45-3.64 (m, 4H), 2.67-2.77 (m, 2H), 2.46-2.58 (m, 2H), 1.45 (d, 3H), 1.28 (t, 3H).

### EXAMPLE 11

### 4-[3-(3-Chloro-phenyl)-1-ethyl-prop-2-ynyl]-piperazine-1-carboxylic acid ethyl ester

Water (2.5 mL) was deoxygenated with argon for 1 minute in a vial. 3-chloro-1-ethynyl-benzene (1.0 g, 7.3 mmol), Ethyl-1-piperizinecarboxylate (0.4 mL, 2.7 mmol), gold (III) bromide (30 mg, 0.03 mmol) and propionaldehyde (0.26 mL, 3.7 mmol) were added, and the reaction heated to 100° C., sealed and stirred for 69 h. Reaction mixture was extracted with EtOAc (40 mL) and washed with brine (10 mL), then dried (Na₂SO₄), filtered and concentrated onto silica gel. Chromatography (SPE) eluting with 0-30% EtOAc/hexanes afforded 0.31 g (34%) of the title compound as a brown oil. ¹H NMR (CDCl₃) δ (ppm): 7.42 (td, 1H), 7.21-7.34 (m, 3H), 4.16 (q, 2H), 3.42-3.62 (m, 5H), 2.64-2.75 (m, 2H), 2.45-2.55 (m, 2H), 1.76 (m, 2H), 1.28 (t, 3H), 1.08 (t, 3H).

### EXAMPLE 12

### 4-[3-(3-Chloro-phenyl)-1-isopropyl-prop-2-ynyl]-piperazine-1-carboxylic acid ethyl ester

Water (2.5 mL) was deoxygenated with argon for 1 minute in a vial. 3-chloro-1-ethynyl-benzene (1.0 g, 7.3 mmol), Ethyl-1-piperizinecarboxylate (0.4 mL, 2.7 mmol), gold (III) bromide (30 mg, 0.03 mmol) and 2-methylpropionaldehyde (0.33 mL, 3.7 mmol) were added, and the reaction heated to 100° C., sealed and stirred for 69 h. Reaction mixture was extracted with EtOAc (40 mL) and washed with brine (10 mL), then dried (Na₂SO₄), filtered and concentrated onto silica gel. Chromatography (SPE) eluting with 0-30% EtOAc/hexanes afforded 0.63 g (66%) of the title compound as a brown oil. ¹H NMR (CDCl₃) δ (ppm): 7.42 (t, 1H), 7.21-7.34 (m, 3H), 4.16 (q, 2H), 3.43-3.61 (m, 4H), 2.60-2.71 (m, 2H), 2.40-2.51 (m, 2H), 1.84-1.98 (m, 1H), 1.28 (t, 3H), 1.12 (d, 3H), 1.04 (d, 3H).

### EXAMPLE 13

### 4-[1-tert-Butyl-3-(3-chloro-phenyl)-prop-2-ynyl]-piperazine-1-carboxylic acid ethyl ester

Water (2.5 mL) was deoxygenated with argon for 1 minute in a vial. 3-chloro-1-ethynyl-benzene (1.0 g, 7.3 mmol), Ethyl-1-piperizinecarboxylate (0.4 mL, 2.7 mmol), gold (III) bromide (30 mg, 0.03 mmol) and 2,2-dimethylpropionaldehyde (0.40 mL, 3.7 mmol) were added, and the reaction heated to 100° C., sealed and stirred for 69 h. Reaction mixture was extracted with EtOAc (40 mL) and washed with brine (10 mL), then dried (Na2SO4), filtered and concentrated onto silica gel. Chromatography (SPE) eluting with 5-30% EtOAc/hexanes afforded 0.19 g (19%) of the title compound as a yellow oil. ¹H NMR (CDCl₃) δ (ppm): 7.42 (td, J=2, 0.5 Hz, 1H), 7.21-7.34 (m, 3H), 4.15 (q, J=7 Hz, 2H), 3.42-3.58 (m, 4H), 3.16 (s, 1H), 2.70-2.80 (m, 2H), 2.48-2.58 (m, 2H), 1.28 (t, J=7 Hz, 3H), 1.05 (s, 9H).

### EXAMPLE 14

### 4-[3-(3-Chloro-phenyl)-1-phenyl-prop-2-ynyl]-piperazine-1-carboxylic acid ethyl ester

Water (2.5 mL) was deoxygenated with argon for 1 minute in a vial. 3-chloro-1-ethynyl-benzene (1.0 g, 7.3 mmol), Ethyl-1-piperizinecarboxylate (0.4 mL, 2.7 mmol), gold (III) bromide (30 mg, 0.03 mmol) and benzaldehyde (0.37 mL, 3.7 mmol) were added, and the reaction heated to 100° C., sealed and stirred for 69 h. Reaction mixture was extracted with EtOAc (40 mL) and washed with brine (10 mL), then dried (Na₂SO₄), filtered and concentrated onto silica gel. Chromatography (SPE) eluting with 5-30% EtOAc/hexanes afforded 0.72 g (69%) of the title compound as a brown oil. ¹H NMR (CDCl₃) δ (ppm): 7.62 (m, 2H), 7.51 (td, 1H), 7.25-7.44 (m, 6H), 4.87 (s, 1H), 4.15 (q, 2H), 3.44-3.59 (m, 4H), 2.59 (t, 4H), 1.28 (t, 3H).

### EXAMPLE 15

### 4-[1-(3-Chloro-phenylethynyl)-butyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and butyraldehyde (0.13 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 20% EtOAc/hexanes afforded 0.14 g (38%) of the title compound as a brown oil. ¹H NMR (CDCl₃) δ (ppm): 7.42 (td, 1H), 7.21-7.33 (m, 3H), 4.16 (q, 2H), 3.46-3.61 (m, 5H), 2.64-2.74 (m, 2H), 2.45-2.55 (m, 2H), 1.40-1.78 (m, 4H), 1.28 (t, 3H), 0.98 (t, 3H).

### EXAMPLE 16

### 4-[1-(3-Chloro-phenylethynyl)-3-methyl-butyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and isovaleraldehyde (0.16 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 20% EtOAc/hexanes afforded 92 mg (23%) of the title compound as a brown oil. ¹H NMR (CDCl₃) δ (ppm): 7.41 (m, 1H), 7.21-7.33 (m, 3H), 4.16 (q, 2H), 3.45-3.68 (m, 5H), 2.64-2.74 (m, 2H), 2.45-2.55 (m, 2H), 1.89 (m, 1H), 1.51-1.72 (m, 2H), 1.28 (t, 3H), 0.98 (t, 6H).

### EXAMPLE 17

### 4-[1-Benzyloxymethyl-3-(3-chloro-phenyl)-prop-2-ynyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and benzyloxyacetaldehyde (0.20 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 20% EtOAc/hexanes afforded 56 mg (12%) of the title compound as a brown oil. ¹H NMR (CDCl₃) δ (ppm): 7.21-7.42 (m, 9H), 4.65 (d, 2H), 4.16 (q, 2H), 3.92 (dd, 1H), 3.48-3.76 (m, 4H), 2.63-2.72 (m, 2H), 2.51-2.60 (m, 2H), 1.28 (t, 3H).

### EXAMPLE 18

### 4-[3-(3-Chloro-phenyl)-1-cyclopropyl-prop-2-ynyl]-piperazine-1-carboxylic acid ethyl ester

Water (1 mL) was deoxygenated with argon for 1 minute in a vial. 3-chloro-1-ethynyl-benzene (0.166 g, 1.2 mmol), Ethyl-1-piperizinecarboxylate (0.226 g, 1.4 mmol), gold (III) bromide (30 mg, 0.17 mmol) and cyclpropanecarboxaldehyde (0.100 mL, 1.4 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 10% EtOAc/hexanes afforded 0.344 g (83%) of the title compound as a brown oil. ¹H-NMR (CDCl₃), δ (ppm): 7.40 (dd, 1H), 7.27 (m, 3H), 4.15 (q, 2H), 3.62 (d, 1H), 3.54 (m, 4H), 3.99 (m, 2H), 2.80 (m, 2H), 2.56 (m, 2H), 1.28 (d,3H), 1.11 (m, 1H), 0.57 (m,3H), 0.42 (m, 1H).

### EXAMPLE 19

### 4-[1-(3-Chloro-phenylethynyl)-pentyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and valeraldehyde (0.16 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 10% EtOAc/hexanes afforded 0.22 g (55%) of the title compound as a brown oil. ¹H NMR (CDCl₃) δ (ppm): 7.42 (td, 1H), 7.21-7.33 (m, 3H), 4.16 (q, 2H), 3.45-3.62 (m, 5H), 2.64-2.74 (m, 2H), 2.45-2.56 (m, 2H), 1.68-1.78 (m, 2H), 1.32-1.58 (m, 4H), 1.28 (t, 3H), 0.95 (t, 3H).

### EXAMPLE 20

### 4-[3-(3-Chloro-phenyl)-1-thiophen-2-yl-prop-2-ynyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and thiophene 2-carbaldehyde (0.14 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 10% EtOAc/hexanes afforded 83 mg (20%) of the title compound as a brown oil. ¹H NMR (CDCl₃) δ (ppm): 7.40 (td, 1H), 7.26-7.34 (m, 3H), 7.23 (dt, 1H), 7.00 (dd, 1H), 5.06 (d, 1H), 4.16 (q, 2H), 3.54 (m, 4H), 2.64 (m, 4H), 1.28 (t, 3H).

### EXAMPLE 21

### 4-[3-(3-Chloro-phenyl)-1-thiophen-3-yl-prop-2-ynyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and thiophene 3-carbaldehyde (0.14 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 10% EtOAc/hexanes afforded 93 mg (22%) of the title compound as a brown oil. ¹H NMR (CDCl₃) δ (ppm): 7.49 (td, 1H), 7.43 (dt, 1H), 7.25-7.36 (m, 3H), 7.24 (dd, 1H), 4.89 (d, 1H), 4.15 (q, 2H), 3.52 (m, 4H), 2.59 (t, 4H), 1.27 (t, 3H).

### EXAMPLE 22

### 4-[3-(3-Chloro-phenyl)-1-furan-2-yl-prop-2-ynyl]-piperazine-1-carboxylic acid ethyl ester

Water (0.5 mL) was deoxygenated with argon for 1 minute in a vial. 3-chloro-1-ethynyl-benzene (0.18 mL, 1.5 mmol), Ethyl-1-piperizinecarboxylate (0.16 mL, 1.1 mmol), gold (III) bromide (6 mg, 0.03 mmol) and thiophene 3-carbaldehyde (0.14 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silica gel. Chromatography (SPE) eluting with 10% EtOAc/hexanes afforded 0.12 g (29%) of the title compound as a brown oil. ¹H NMR (CDCl₃) δ (ppm): 7.49 (td, 1H), 7.46 (dd, 1H), 7.25-7.40 (m, 3H), 6.51 (dt, 1H), 6.39 (dd, 1H), 4.15 (q, 2H), 4.94 (s, 1H), 3.56 (m, 4H), 2.62 (m, 4H), 1.27 (t, 3H).

### EXAMPLE 23

### 4-[3-(3-Chloro-phenyl)-1-ethyl-prop-2-ynyl]-piperazine-1-carboxylic acid tert-butyl ester

Water (0.5 mL) was deoxygenated with argon for 1 minute in a vial. 3-chloro-1-ethynyl-benzene (0.18 mL, 1.5 mmol), piperazine-1-carboxylic acid tert-butyl ester (0.20 g, 1.1 mmol), gold (III) bromide (6 mg, 0.03 mmol) and propionaldehyde (0.10 mL, 1.5 mmol) were added, and the reaction heated to 100° C., sealed and stirred for 16 h. Reaction mixture was extracted with EtOAc (40 mL) and washed with brine (10 mL), then dried (Na₂SO₄), filtered and concentrated onto silicate. Chromatography (SPE) eluting with 30% EtOAc/hexanes afforded 11 mg (3%) of the title compound as a brown oil. ¹H NMR (CDCl₃) δ (ppm): 7.42 (m, 1H), 7.21-7.35 (m, 3H), 3.40-3.56 (m, 5H), 2.62-2.73 (m, 2H), 2.37-2.55 (m, 2H), 1.76 (m, 2H), 1.48 (s, 9H), 1.08 (t, 3H).

### EXAMPLE 24

### 1-[3-(3-Chloro-phenyl)-1-ethyl-prop-2-ynyl]-piperazine

4-[3-(3-Chloro-phenyl)-1-ethyl-prop-2-ynyl]-piperazine-1-carboxylic acid tert-butyl ester (0.29 g, 0.78 mmol) was dissolved in CH₂Cl₂ (1 mL) and cooled to 0° C. TFA (1 mL, 13.5 mmol) was added slowly, and the reaction stirred for 45 minutes while warming to room temperature. Reaction mixture was poured into a saturated NaHCO₃ solution (30 mL), extracted with CH₂Cl₂ (2×40 mL), then dried (Na₂SO₄), filtered and concentrated onto silica gel. Chromatography (SPE) eluting with 80% EtOAc/hexanes followed by 15-20% 2.0 M NH₃ in MeOH/EtOAc afforded 0.17 g (83%) of the title compound as a yellow oil. ¹H NMR (CDCl₃) δ (ppm): 7.44 (td, 1H), 7.34 (dt, 1H), 7.21-7.29 (m, 2H), 4.48 (bs, 1H), 3.43 (t, 1H), 3.00-3.16 (m, 4H), 2.76-2.87 (m, 2H), 2.55-2.70 (m, 2H), 1.74 (m, 2H), 1.07 (t, 3H).

### EXAMPLE 25

### 4-[3-(3-Chloro-phenyl)-1-ethyl-prop-2-ynyl]-piperazine-1-carboxylic acid isopropyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Isopropyl chloroformate (1.0 M solution, 0.23 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 19 mg (36%) of the title compound as a yellow oil. ¹H NMR (CDCl₃) δ (ppm): 7.42 (td, 1H), 7.21-7.33 (m, 3H), 4.94 (7, 1H), 3.42-3.60 (m, 5H), 2.64-2.74 (m, 2H), 2.47-2.56 (m, 2H), 1.76 (m, 2H), 1.26 (d, 6H), 1.08 (t, 3H).

### EXAMPLE 26

### 4-[3-(3-Chloro-phenyl)-1-ethyl-prop-2-ynyl]-piperazine-1-carboxylic acid propyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. N-propyl chloroformate (0.027 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 11 mg (20%) of the title compound as a yellow oil. ¹H NMR (CDCl₃) δ (ppm): 7.42 (td, 1H), 7.21-7.33 (m, 3H), 3.88 (d, 2H), 3.43-3.62 (m, 5H), 2.64-2.74 (m, 2H), 2.46-2.55 (m, 2H), 2.46-2.55 (m, 2H), 1.61-1.82 (m, 4H), 0.96 (t, 3H).

### EXAMPLE 27

### 4-[3-(3-Chloro-phenyl)-1-ethyl-prop-2-ynyl]-piperazine-1-carboxylic acid isobutyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Isobutyl chloroformate (0.030 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 24 mg (44%) of the title compound as a yellow oil. ¹H NMR (CDCl₃) δ (ppm): 7.42 (td, 1H), 7.21-7.33 (m, 3H), 3.88 (d, 2H), 3.43-3.62 m, 5H), 2.65-2.74 (m, 2H), 2.46-2.56 (m, 2H), 1.95 (m, 1H), 1.76 (m, 2H), 1.08 (t, 3H), 0.95 (d, 6H).

### EXAMPLE 29

### 4-[3-(3-Chloro-phenyl)-1-ethyl-prop-2-ynyl]-piperazine-1-carboxylic acid butyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. N-butyl chloroformate (0.029 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 20 mg (37%) of the title compound as a yellow oil. ¹H NMR (CDCl₃) δ (ppm): 7.42 (m, 1H), 7.21-7.33 (m, 3H), 4.10 (t, 2H), 3.42-3.61 (m, 5H), 2.64-2.75 (m, 2H), 2.46-2.56 (m, 2H), 1.58-1.81 (m, 4H), 1.32-1.47 (m, 2H), 1.08 (t, 3H), 0.95 (t, 3H).

### EXAMPLE 30

### 4-[3-(3-Chloro-phenyl)-1-ethyl-prop-2-ynyl]-piperazine-1-carboxylic acid 2,2-dimethyl-propyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Neopentyl chloroformate (0.034 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 26 mg (46%) of the title compound as a yellow oil. ¹H NMR (CDCl₃) δ (ppm): 7.42 (m, 1H), 7.21-7.33 (m, 3H), 3.81 (s, 2H), 3.43-3.62 (m, 5H), 2.64-2.75 (m, 2H), 2.45-2.58 (m, 2H), 1.76 (m, 2H), 1.08 (t, 3H), 0.96 (s, 9H).

### EXAMPLE 31

### 4-[3-(3-Chloro-phenyl)-1-ethyl-prop-2-ynyl]-piperazine-1-carboxylic acid pentyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. N-pentyl chloroformate (0.033 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 26 mg (45%) of the title compound as a yellow oil. ¹H NMR (CDCl₃) δ (ppm): 7.42 (m, 1H), 7.21-7.33 (m, 3H), 4.09 (t, J=7 Hz, 2H), 3.42-3.61 (m, 5H), 2.64-2.74 (m, 2H), 2.45-2.55 (m, 2H), 1.57-1.81 (m, 4H), 1.24-1.38 (m, 4H), 1.08 (t, J=7 Hz, 3H), 0.84-0.96 (m, 3H).

### EXAMPLE 32

### 4-[3-(3-Chloro-phenyl)-1-ethyl-prop-2-ynyl]-piperazine-1-carboxylic acid 2-methoxy-ethyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Chloroformic acid 2-methoxyethyl ester (0.027 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 70% EtOAc/hexanes afforded 15 mg (27%) of the title compound as a colourless oil. ¹H NMR (CDCl₃) δ (ppm): 7.42 (td, 1H), 7.21-7.33 (m, 3H), 4.26 (m, 2H), 3.42-3.64 (m, 7H), 3.40 (s, 3H), 2.64-2.74 (m, 2H), 2.46-2.56 (m, 2H), 1.76 (m, 2H), 1.08 (t, 3H).

### EXAMPLE 33

### 4-[3-(3-Chloro-phenyl)-1-ethyl-prop-2-ynyl]-piperazine-1-carboxylic acid phenyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Phenyl chloroformate (0.029 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 18 mg (30%) of the title compound as a yellow oil. ¹H NMR (CDCl₃) δ (ppm): 7.45 (m, 1H), 7.11-7.41 (r, 8H), 3.58-3.79 (m, 4H), 3.46 (t, 1H), 7.74-2.83 (m, 2H), 2.56-2.64 (m, 2H), 1.79 (m, 2H), 1.10 (t, 3H).

### EXAMPLE 34

### 4-[3-(3-Chloro-phenyl)-1-ethyl-prop-2-ynyl]-piperazine-1-carboxylic acid benzyl ester

1-[3-(3-Chloro-phenyl)-1-ethyl-prop-2-ynyl]-piperazine (40 mg, 0.15 mmol) and triethylamine (0.064 mL, 0.46 mmol) were dissolved in CH₂Cl₂ (˜2 mL) and stirred at room temperature. Benzyl chloroformate (0.033 mL, 0.23 mmol) was added, and the reaction stirred at room temperature for 3 h. Excess triethylamine/CH₂Cl₂ were evaporated, and the residue was taken up in EtOAc (15 mL) and washed with water (3×10 mL) and brine (10 mL). Chromatography (SPE) eluting with 30% EtOAc/hexanes afforded 26 mg (43%) of the title compound as a yellow oil. ¹H NMR (CDCl₃) δ (ppm): 7.21-7.43 (m, 9H), 5.16 (s, 2H), 3.50-3.65 (m, 4H), 3.46 (t, 1H), 2.64-2.76 (m, 2H), 2.46-2.58 (m, 2H), 1.75 (m, 2H), 1.08 (t, 3H).

### EXAMPLE 35

### 4-[3-(3-Chloro-phenyl)-1-pyridin-3-yl-prop-2-ynyl]-piperazine-1-carboxylic acid ethyl ester

3-chloro-1-ethynyl-benzene (0.345 mL, 2.80 mmol), ethyl-1-piperizinecarboxylate (0.301 mL, 2.05 mmol), gold (III) bromide (8.2 mg, 0.018 mmol), pyridine-3-carbaldehyde (0.176 mL, 1.87 mmol) and deoxygenated water (1.9 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 5-50% ethyl acetate in hexanes afforded the titled compound (101.8 mg, 14%, yellow oil). ¹H NMR (CDCl₃) δ (ppm): 8.87 (m, 1H), 8.59 (m, 1H), 7.92 (m, 1H), 7.50 (m, 4H), 7.34 (m, 1H), 4.91 (s, 1H), 4.14 (q, 2H), 3.54 (m, 4H), 2.58 (m, 4H), 1.27 (t, 3H).

### EXAMPLE 36

### 4-[3-(3-Chloro-phenyl)-1-(2,4-difluoro-phenyl)-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 2,4-difluoro-benzaldehyde (0.081 mL, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 4-10% ethyl acetate in hexanes afforded the titled compound (107.2 mg, 35%, yellow oil). ¹H NMR (CDCl₃) δ (ppm): 7.62 (m, 1H), 7.48 (m, 1H), 7.33 (m, 3H), 6.89 (m, 2H), 5.09 (s, 1H), 4.14 (q, 2H), 3.49 (m, 4H), 2.59 (m, 4H), 1.27 (t, 3H).

### EXAMPLE 37

### 4-[3-(3-Chloro-phenyl)-1-(2-methoxy-phenyl)-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 2-methoxy-benzaldehyde (0.090 mL, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 4-10% ethyl acetate in hexanes afforded the titled compound (232.2 mg, 76%, yellow oil). ¹H NMR (CDCl₃) δ (ppm): 7.60 (m, 1H), 7.46 (m, 1H), 7.31 (m, 4H), 6.98 (m, 2H), 5.26 (s, 1H), 4.14 (q, 2H), 3.89 (s, 3H), 3.51 (m, 4H), 2.63 (m, 4H), 1.26 (t, 3H).

### EXAMPLE 38

### 4-[3-(3-Chloro-phenyl)-1-(2-chloro-phenyl)-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 2-chloro-benzaldehyde (103.5 mg, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 4-10% ethyl acetate in hexanes afforded the titled compound (202.3 mg, 66%, yellow oil). ¹H NMR (CDCl₃) δ (ppm): 7.71 (m, 1H), 7.49 (m, 1H), 7.35 (m, 6H), 5.12 (s, 1H), 4.15 (q, 2H), 3.47 (m, 4H), 2.63 (m, 4H), 1.27 (t, 3H).

### EXAMPLE 39

### 4-[3-(3-Chloro-phenyl)-1-o-tolyl-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 2-methyl-benzaldehyde (0.086 mL, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 2-10% ethyl acetate in hexanes afforded the titled compound (151.1 mg, 51%, yellow oil). ¹H NMR (CDCl₃) δ (ppm): 7.50 (m, 1H), 7.39 (m, 1H), 7.28 (m, 6H), 4.93 (s, 1H), 4.15 (q, 2H), 3.46 (m, 4H), 2.58 (m, 4H), 2.48 (s, 3H), 1.27 (t, 3H).

### EXAMPLE 40

### 4-[3-(3-Chloro-phenyl)-1-m-tolyl-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 3-methyl-benzaldehyde (0.087 mL, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 2-10% ethyl acetate in hexanes afforded the titled compound (165 mg, 56%, yellow oil). ¹H NMR (CDCl₃) δ (ppm): 7.51 (m, 1H), 7.33 (m, 6H), 7.15 (m, 1H), 4.82 (s, 1H), 4.15 (q, 2H), 3.54 (m, 4H), 2.59 (m, 4H), 2.41 (s, 3H), 1.27 (t, 3H).

### EXAMPLE 41

### 4-[3-(3-Chloro-phenyl)-1-(6-methoxy-pyridin-3-yl)-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 6-methoxy-pyridine-3-carbaldehyde (101.5 mg, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 5-20% ethyl acetate in hexanes afforded the titled compound (138.8 mg, 45%, yellow oil). ¹H NMR (CDCl₃) δ (ppm): 8.40 (m, 1H), 7.81 (m, 1H), 7.50 (m, 1H), 7.33 (m, 3H), 6.78 (m, 1H), 4.82 (s, 1H), 4.15 (q, 2H), 3.97 (s, 3H), 3.52 (m, 4H), 2.58 (m, 4H), 1.27 (t, 3H).

### EXAMPLE 42

### 4-[3-(3-Chloro-phenyl)-1-(2-chloro-pyridin-3-yl)-prop-2-ynyl]-piperazine-1-carboxylic acid ethyl ester

3-chloro-1-ethynyl-benzene (0.136 mL, 1.10 mmol), ethyl-1-piperizinecarboxylate (0.119 mL, 0.81 mmol), gold (III) bromide (3.2 mg, 0.0074 mmol), 2-chloro-pyridine-3-carbaldehyde (104.8 mg, 0.74 mmol) and deoxygenated water (0.8 mL) were added to a vial, sealed, and stirred at 100° C. overnight. The reaction mixture was cooled and then extracted with dichloromethane, washed with water, dried over anhydrous sodium sulfate, filtered and concentrated onto silica gel. Purification chromatography (SPE) using 5-20% ethyl acetate in hexanes afforded the titled compound (169.7 mg, 55%, yellow oil). ¹H NMR (CDCl₃) δ (ppm): 8.40 (m, 1H), 8.04 (m, 1H), 7.48 (m, 1H), 7.34 (m, 4H), 5.12 (s, 1H), 4.15 (q, 2H), 3.49 (m, 4H), 2.61 (m, 4H), 1.28 (t, 3H).

### EXAMPLE 43

### (S)-3-Methyl-piperazine-1-carboxylic acid ethyl ester

(S)-2-methyl-piperazine (500 mg, 4.99 mmol) was dissolved with stirring in dichloromethane (2.5 mL) and the solution was cooled to 0° C. Ethyl chloroformate (239 μL, 2.49 mmol) was added via a syringe drop wise. The mixture was allowed to warm to room temperature and stirred for 3 h. When TLC analysis showed that the reaction was complete, the mixture was diluted with dichloromethane and washed with water. The organic phase was dried (Na₂SO₄), filtered and concentrated to yield the title compound, a yellowish liquid (315.8 mg, 73%). ¹H NMR (300 MHz, CDCl₃) δ=0.70 (d, J=6.3 Hz, 3H); 0.91 (t, J=7 Hz, 3H); 1.42 (s, broad, 1H); 2.06 (s, broad, 1H); 2.36 (m, 3H); 2.61 (m, 1H); 3.64 (s, broad, 2H); 3.78 (q, J=7 Hz, 2H).

### EXAMPLE 44

### (R)-3-Methyl-piperazine-1-carboxylic acid ethyl ester

The title compound was made from (R)-2-methyl-piperazine in the same manner as the (S)-enantiomer above.

### EXAMPLE 45

General Procedure: Gold Catalyzed Coupling of Amine, Aldehyde and Alkyne piperazine (1 mmol), and gold (III) bromide (0.01 mmol) were weighed into a vial. Alkyne (1.35 mmol) and aldehyde (1.35 mmol) were added followed by deionized water (1.35 mL). The vial was capped and the reaction mixture was stirred overnight at 100° C. The reaction mixture was then diluted with deionized water and organic products were extracted with dichloromethane three times. The organic phase was dried (Na₂SO₄), filtered and concentrated onto silica gel. Chromatography (SPE column using 20-50% ethyl acetate in hexanes) yielded the product.

The following compounds were made in this manner:

a) Ethyl 4-[3-(5-chloro-2-fluorophenyl)-1-ethylprop-2-yn-1-yl]piperazine-1-carboxylate; yield 7%, yellow oil; ¹H NMR (300 MHz, CDCl₃) δ: 1.08 (t, J=7.5 Hz, 3H); 1.28 (t, 3.6 Hz, 3H); 1.75 (m, 2H); 2.51 (m, 2H); 2.69 (m, 2H); 3.54 (m, 5H); 4.16 (q, J=14.1, 6.9 Hz, 2H); 7.02 (t, J=8.7 Hz, 1H); 7.24 (m, 1H); 7.40 (dd, J=6, 2.7 Hz, 1H).

b) Ethyl 4-[3-(3-chlorophenyl)-1-(5-methyl-2-furyl)prop-2-yn-1-yl]piperazine-1-carboxylate; ¹H NMR (300 MHz, CDCl₃) δ: 1.25 (t, J=7 Hz, 3H); 2.31 (s, 3H); 2.60 m, 4H); 3.53 (m, 4H); 4.13 (q, J=7 Hz, 2H); 4.85 (s, 1H); 5.94 (d, J=3 Hz, 1H); 6.36 (d, J=3 Hz, 1H); 7.32 (m, 3H); 7.46 (s, 1H).

c) Ethyl 4-{3-(3-chlorophenyl)-1-[5-(methoxycarbonyl)-2-furyl]prop-2-yn-1-yl}piperazine-1-carboxylate; ¹H NMR (300 MHz, CDCl₃) δ: 1.23 (t, J=7 Hz, 3H); 2.58 (m, 4H); 3.49 (m, 4H); 3.79 (s, 3H); 4.10 (q, J=7 Hz, 2H); 4.83 (s, 1H); 6.70 (s, 1H); 7.29 (m, 4H); 7.44 (m, 1H).

d) 2,2,2-Trifluoroethyl 4-[3-(3-chlorophenyl)-1-(2-furyl)prop-2-yn-1-yl]piperazine-1-carboxylate; ¹H NMR (300 MHz, CDCl₃) δ: 2.65 (m, 4H); 3.60 (m, 4H); 4.50 (q, J=8.5 Hz, 2H), 4.95 (s, 1H); 6.40 (m, 1H); 6.51 (m, 1H); 7.34 (m, 3H); 7.48 (m, 2H).

e) Ethyl 4-{3-(3-chlorophenyl)-1-[5-(hydroxymethyl)-2-furyl]prop-2-yn-1-yl}piperazine-1-carboxylate; ¹H NMR (300 MHz, CDCl₃) δ: 1.26 (t, J=7 Hz, 3H); 2.61 (m, 4H); 3.55 (m, 4H); 4.13 (q, J=7 Hz, 2H); 4.62 (s, 2H); 4.90 (s, 1H); 6.28 (d, J=3.3 Hz, 1H); 6.45 (d, J=3.3 Hz, 1H); 7.32 (m, 3H); 7.47 (m, 1H).

f) Ethyl (3S)-4-[(1R)-3-(3-chlorophenyl)-1-(2-furyl)prop-2-yn-1-yl]-3-methylpiperazine-1-carboxylate; yield 3.7% pure fraction; ¹H NMR (300 MHz, CDCl₃) δ: 1.26 (m, 6H); 2.33 (m, 1H); 2.55 (m, 1H); 2.89 (m, 1H); 3.20 (m, 2H); 3.91 (m, 2H); 4.13 (m, 2H); 5.28 (s, 1H); 6.39 (m, 1H); 6.41 (m, 1H); 7.32 (m, 3H); 7.43 (m, 1H); 7.48 (m, 1H).

g) Ethyl (3S)-4-[(1S)-3-(3-chlorophenyl)-1-(2-furyl)prop-2-yn-1-yl]-3-methylpiperazine-1-carboxylate; yield 15.3% pure fraction; ¹H NMR (300 MHz, CDCl₃) δ: 1.29 (m, 6H); 2.45 (m, 2H); 2.71 (m, 1H); 2.81 (m, 1H); 2.94 (m, 1H); 3.99 (m, 2H); 4.14 (m, 2H); 5.34 (s, 1H); 6.38 (m, 1H); 6.54 (m, 1H); 7.33 (m, 3H); 7.46 (m, 2H).

h) Ethyl (3R)-4-[(1S)-3-(3-chlorophenyl)-1-ethylprop-2-yn-1-yl]-3-methylpiperazine-1-carboxylate; yield 18.5% pure fraction; ¹H NMR (300 MHz, CDCl₃) δ: 1.06 (t, J=7.2 Hz, 3H); 1.10 (d, J=6.0 Hz, 3H); 1.27 (t, J=7.1 Hz, 3H); 1.73 (m, 2H); 2.39 (m, 1H); 2.60 (m, 2H); 2.77 (m, 1H); 2.90 (m, 1H); 3.81 (m, 1H); 3.95 (m, 2H); 4.14 (q, 7.2, 2H); 7.26 (m, 3H); 7.40 (s, 1H).

i) Ethyl (3R)-4-[(1R)-3-(3-chlorophenyl)-1-(2-furyl)prop-2-yn-1-yl]-3-methylpiperazine-1-carboxylate, yield 3.7% pure fraction; ¹H NMR (300 MHz, CDCl₃) δ: 1.29 (m, 6H); 2.45 (m, 2H); 2.71 (m, 1H); 2.81 (m, 1H); 2.94 (m, 1H); 3.99 (m, 2H); 4.14 (m, 2H); 5.34 (s, 1H); 6.38 (m, 1H); 6.54 (m, 1H); 7.33 (m, 3H); 7.46 (m, 2H).

j) Ethyl (3R)-4-[(1R)-3-(3-chlorophenyl)-1-ethylprop-2-yn-1-yl]-3-methylpiperazine-1-carboxylate; yield 5.5% pure fraction; ¹H NMR (300 MHz, CDCl₃) δ: 1.06 (t, J=7.3 Hz, 3H); 1.14 (d, J=6.3 Hz, 3H); 1.28 (t, J=7.4 Hz, 3H); 1.70 (m, 2H); 2.58 (m, 1H); 2.75 (m, 1H); 3.08 (m, 2H), 3.40 (m, 1H); 3.66 (m, 3H); 4.15 (q, 7.4, 2H); 7.27 (m, 3H); 7.42 (s, 1H).

k) Ethyl (3S)-4-[(1S)-3-(3-chlorophenyl)-1-ethylprop-2-yn-1-yl]-3-methylpiperazine-1-carboxylate; yield 7.5% pure fraction; ¹H NMR (300 MHz, CDCl₃) δ: 1.06 (t, J=7.3 Hz, 3H); 1.14 (d, J=6.3 Hz, 3H); 1.28 (t, J=7.4 Hz, 3H); 1.70 (m, 2H); 2.58 (m, 1H); 2.75 (m, 1H); 3.08 (m, 2H), 3.40 (m, 1H); 3.66 (m, 3H); 4.15 (q, 7.4, 2H); 7.27 (m, 3H); 7.42 (s, 1H).

l) Ethyl (3S)-4-[(1R)-3-(3-chlorophenyl)-1-methylprop-2-yn-1-yl]-3-methylpiperazine-1-carboxylate; yield 30.5% pure fraction; ¹H NMR (300 MHz, CDCl₃) δ: 1.06 (t, J=7.2 Hz, 3H); 1.10 (d, J=6.0 Hz, 3H); 1.27 (t, J=7.1 Hz, 3H); 1.73 (m, 2H); 2.39 (m, 1H); 2.60 (m, 2H); 2.77 (m, 1H); 2.90 (m, 1H); 3.81 (m, 1H); 3.95 (m, 2H); 4.14 (q, 7.2, 2H); 7.26 (m, 3H); 7.40 (s, 1H).

### EXAMPLE 46

### 4-[3-(3-Chloro-phenyl)-prop-2-ynyl]-piperazine-1-carboxylic acid tert-butyl ester

Piperazine-1-carboxylic acid tert-butyl ester (500 mg) was added to a mixture of 1-chloro-3-iodo-benzene (51.9 μL, 0.4184 mmol), 3-bromo-propyne (44.7 μL, 0.502 mmol), copper (I) iodide (7.96 mg, 0.0209 mmol) and bis(triphenylphosphine)-palladium(II) dichloride (14.68 mg, 0.04184 mmol) in a screw cap vial. The reaction mixture was heated to 60° C. A small amount of dichloromethane was added to dissolve/melt the piperazine solvent. When TLC analysis showed that the reaction was complete, the mixture was diluted with dichloromethane and washed with water. The aqueous phase was re-extracted with dichloromethane. The combined organics were dried (Na₂SO₄), filtered and chromatographed in 30-50% ethyl acetate in hexanes to yield the title compound (106.6 mg, 76%). ¹H NMR (300 MHz, CDCl₃) δ=1.47 (s, 9H); 2.57 (t, J=4.8 Hz, 4H); 3.51 (m, 6H); 7.27 (m, 3H); 7.42 (s, 1H).

### EXAMPLE 47

### 1-[3-(3-Chloro-phenyl)-prop-2-ynyl]-piperazine

4-[3-(3-Chloro-phenyl)-prop-2-ynyl]-piperazine-1-carboxylic acid tert-butyl ester (106 mg) was dissolved in dichloromethane (1 mL) with stirring. A solution of trifluoroacetic acid (1 mL) in dichloromethane (1 mL) was added and the reaction was stirred for 1 h. When TLC analysis showed that the reaction was complete, the mixture was diluted with dichloromethane. A small volume of water was added and the trifluoroacetic acid was neutralized with solid sodium bicarbonate. The organic phase was separated, and the aqueous phase was re-extracted after basifying with addition of 1M NaOH. The combined organics were dried (Na₂SO₄), filtered and concentrated under reduced pressure to afford in quantitative yield the desired product, pure by TLC and NMR. ¹H NMR (300 MHz, CDCl₃) δ=2.75 (m, 4H); 3.12 (m, 4H); 3.53 (s, 2H); 6.71 (b, 1H); 7.28 (m, 3H); 7.42 (m, 1H).

### EXAMPLE 48

Ethoxy-acetaldehyde

To a cooled, stirred solution of oxalyl chloride (16.6 mL of 2M sol, 33.3 mmol) in dichloromethane (20 mL) was added dimethylsulfoxide (3.7 mL, 52.6 mmol) dropwise. After the solution was stirred for 10 min, 2-ethoxy-ethanol (1.075 mL, 11.1 mmol in 10 mL of dichloromethane) was added dropwise. After the solution was stirred for another 30 min, triethylamine (13.45 mL, 96.5 mmol) was added. The reaction mixture was allowed to warm to room temperature and the organic phase was separated. The aqueous phase was extracted again with dichloromethane. The combined organics were dried (Na₂SO₄), filtered and concentrated onto silica gel. Chromatography on silica gel in 10% ethyl acetate in hexanes yielded the product. ¹H NMR (300 MHz, CDCl₃) δ=1.37 (t, J=7 Hz, 3H); 3.85 (q, J=7 Hz, 2H); 5.04 (d, J=2.7 Hz, 1H); 5.17 (d, J=2.7 Hz, 1H); 9.25 (s, 1H).

### EXAMPLE 49

General Procedure: Copper Catalyzed Coupling of Amine, Aldehyde and Alkyne Acetylene (1.35 mmol), aldehyde (1.35 mmol), piperazine (1 mmol) and copper (I) iodide (0.15 mmol) was added to a microwave safe reaction vessel. Water (1.25 mL) was added with a stir bar, and the mixture was stirred for 5 min with heating at 170° C. in a microwave reactor. The reaction mixture was then diluted with dichloromethane and washed with water. The organic phase was dried (Na₂SO₄), filtered and concentrated onto silica gel. Chromatography in 30-60% ethyl acetate in hexanes yielded the desired compound.

The following compounds were made in this manner:

a) 4-[1-(tert-Butoxycarbonylamino-methyl)-3-(3-chloro-phenyl)-prop-2-ynyl]-piperazine-1-carboxylic acid ethyl ester; yield 16%; ¹H NMR (300 MHz, CDCl₃): 1.27 (t, J=7 Hz, 3H); 1.48 (s, 9H); 2.51 (m, 2H); 2.68 (m, 2H); 3.33 (m, 1H); 3.52 (m, 5H); 3.69 (m, 1H); 4.15 (q, J=7 Hz, 2H); 5.31 (s, broad, 1H); 7.27 (m, 3H); 7.41 (m, 1H).

b) 4-[3-(3-Chloro-phenyl)-1-triisopropylsilyloxymethyl-prop-2-ynyl]-piperazine-1-carboxylic acid ethyl ester; ¹H NMR (300 MHz, CDCl₃):1.08 (m, 21H); 1.27 (t, J=7.1 Hz, 3H); 2.61 (m, 2H); 2.71 (m, 2H); 3.52 (m, 4H); 3.74 (t, J=6.3 Hz, 1H); 3.96 (d, J=6.3 Hz, 2H); 4.14 (q, J=7.1 Hz, 2H); 3.28 (m, 3H); 7.41 (m, 1H).

c) ethyl 4-[3-(3-chlorophenyl)-1-(ethoxymethyl)prop-2-yn-1-yl]piperazine-1-carboxylate; ¹H NMR (300 MHz, CDCl₃): 1.25 (t, J=7.5 Hz, 3H); 1.28 (t, J=7.4 Hz, 3H); 2.60 (m, 2H); 2.69 (m, 2H); 3.55 (m, 4H); 3.64 (m, 3H); 3.71 (m, 1H); 3.88 (t, J=6.3 Hz, 1H); 4.16 (q, J=7.2 Hz, 2H); 7.31 (m, 3H); 7.43 (m, 1H).

### EXAMPLE 50

### 4-[1-aminomethyl)-3-(3-chloro-phenyl)-prop-2-ynyl]-piperazine-1-carboxylic acid ethyl ester

A solution of trifluoroacetic acid (1 mL) in dichloromethane (0.5 mL) was added to a stirred solution of 4-[1-(tert-Butoxycarbonylamino-methyl)-3-(3-chloro-phenyl)-prop-2-ynyl]-piperazine-1-carboxylic acid ethyl ester (˜50 mg) in dichloromethane (0.5 mL). The solution was stirred for 30 min. When TLC analysis showed that the reaction was complete, the mixture was diluted with dichloromethane, washed with a small amount of water, and neutralized with solid sodium bicarbonate. The organic phase was dried (Na₂SO₄) filtered and concentrated to yield the desired compound (30.1 mg, 78%). ¹H NMR (300 MHz, CDCl₃) δ=1.28 (t, J=7 Hz, 3H); 2.19 (d, J=1 Hz, 2H); 2.58 (m, 2H); 2.73 (m, 2H); 3.54 (m, 5H); 4.16 (q, J=7 Hz, 2H); 7.27 (m, 3H); 7.42 (m, 1H).

### EXAMPLE 51

### 1,4-Bis-triisopropylsilyloxy-but-2-ene

To a solution of but-2-ene-1,4-diol (0.934 mL, 11.4 mmol) in DMF (15 mL) was added imidazole (1.93 g, 28.4 mmol), followed by chloro-triisopropyl-silane (6.07 mL, 28.4 mmol). The reaction was stirred overnight at room temperature. When TLC analysis showed that the reaction was complete, the mixture was diluted with dichloromethane and washed with water. The organic phase was dried (Na₂SO₄), filtered and concentrated onto silica gel, then chromatographed in (0-10%) ethyl acetate in hexanes to yield the product (3.51 g, 77%). ¹H NMR (300 MHz, CDCl₃) δ=1.09 (m, 42H); 4.32 (dd, J=3.3, 0.6 Hz, 4H); 5.60 (t, J=0.6 Hz, 2H).

### EXAMPLE 52

### Triisopropylsilyloxy-acetaldehyde

1,4-Bis-triisopropylsilyloxy-but-2-ene (3 g, 7.48 mmol) was dissolved in dichloromethane (6 mL) and cooled to −78 C. Ozone was bubbled through the solution until a light blue colour was observed. Oxygen was bubbled through the solution and dimethyl sulfide (5 mL) was added. The reaction was then allowed to warm to room temperature. The mixture was diluted with dichloromethane and washed with water. The organic phase was dried (Na₂SO₄), filtered and concentrated onto silica gel. Chromatography in hexanes yielded the product (3.38 g, 61%). ¹H NMR (300 MHz, CDCl₃) δ=1.08 (m, 21H); 4.28 (d, J=0.9 Hz, 2H); 9.76 (t, J=0.9 Hz, 1H).

### EXAMPLE 53

### 4-[3-(3-Chloro-phenyl)-1-hydroxymethyl-prop-2-ynyl]-piperazine-1-carboxylic acid ethyl ester

4-[3-(3-Chloro-phenyl)-1-triisopropylsilyloxymethyl-prop-2-ynyl]-piperazine-1-carboxylic acid ethyl ester (92.7 mg, 0.173 mmol) was dissolved in THF (0.81 mL). Tetrabutylammonium fluoride (0.189 mL, 1M solution in THF, 0.189 mmol) was added to the solution and stirred for 10 min. When TLC analysis showed that the reaction was complete, the reaction was diluted with dichloromethane and washed with water. The organic phase was dried (Na₂SO₄), filtered and concentrated in vacuo. Chromatography in ethyl acetate to yielded the product (26.9 mg). ¹H NMR (300 MHz, CDCl₃) δ=1.28 (t, J=7.1 Hz, 3H); 2.55 (m, 2H); 2.75 (m, 2H); 3.55 (m, 4H); 3.70 (m, 2H); 3.78 (m, 1H); 4.15 (q, J=7.1 Hz); 7.29 (m, 3H); 7.41 (m, 1H).

### EXAMPLE 54

### 4-[3-(3-Chloro-phenyl)-1-methoxymethyl-prop-2-ynyl]-piperazine-1-carboxylic acid ethyl ester

4-[3-(3-Chloro-phenyl)-1-hydroxymethyl-prop-2-ynyl]-piperazine-1-carboxylic acid ethyl ester (20 mg, 0.0594 mmol) was dissolved in THF (1 mL), and added to a mixture of sodium hydride (3.56 mg, 60% dispersion, 0.0891 mmol) in THF (1 mL). The mixture was stirred for 30 min, and then methyl iodide (3.88 μL, 0.0623 mmol) was added. The solution was then stirred for another 60 min. When TLC analysis showed that the reaction was complete, the solution was diluted with dichloromethane and washed with water. The organic phase was dried (Na₂SO₄), filtered and concentrated under reduced pressure. Chromatography in 50% ethyl acetate in hexanes yielded the product (8.2 mg). ¹H NMR (300 MHz, CDCl₃) δ=1.27 (t, J=7.1 Hz, 3H); 2.58 (m, 2H); 2.69 (m, 2H); 3.45 (s, 3H); 3.57 (m, 5H); 3.70 (m, 1H); 3.90 (m, 1H); 4.15 (q, J=7.1 Hz); 7.28 (m, 3H); 7.43 (m, 1H).

### EXAMPLE 55

### 4-(3-Phenyl-propynoyl)-piperazine-1-carboxylic acid ethyl ester

Phenyl-propynoic acid (50 mg, 0.342 mmol), EDCI (65.58 mg, 0.342 mmol), dimethylaminopyridine (2.78 mg, 0.023 mmol) and piperazine-1-carboxylic acid ethyl ester (36.73 μL, 0.251 mmol) were combined in a screw cap vial and dissolved in dimethylformamide (2 mL). The reaction was stirred overnight at room temperature. The solution was then diluted with dichloromethane and washed with water. The organic phase was dried (Na₂SO₄), filtered and concentrated in vacuo. Chromatography in 0→50% ethyl acetate in hexanes yielded the product (67.6 mg, 94%). ¹H NMR (300 MHz, CDCl₃) δ=1.29 (t, J=7.1 Hz, 3H); 3.51 (m, 2H); 3.58 (m, 2H); 3.69 (m, 2H); 3.83 (m, 2H); 4.17 (q, J=7.1 Hz, 2H); 7.41 (m, 3H); 7.55 (m, 2H).

### EXAMPLE 56

### 4-(1,1-Dimethyl-prop-2-ynyl)-piperazine-1-carboxylic acid ethyl ester

3-Chloro-3-methyl-but-1-yne (1.09 mL, 9.75 mmol) and piperazine-1-carboxylic acid ethyl ester (1.08 mL, 7.39 mmol) were added to a solution of triethylamine (1.38 mL, 9.89 mmol) in THF (10 mL). The solution was stirred vigorously while copper (I) chloride (58.5 mg, 0.59 mmol) was added. An exotherm was observed, as well as a large amount of precipitate, immediately after the addition. The reaction was stirred for 45 min, after which it was diluted with dichloromethane and washed with water. The organic layer was dried, filtered and concentrated, then chromatographed in dichloromethane followed by ethyl acetate to yield the desired product (506.4 mg, 30%). ¹H NMR (300 MHz, CDCl₃) δ=1.28 (t, J=7.2 Hz, 3H); 1.41 (s, 6H); 2.31 (s, 1H); 2.61 (m, 4H); 3.52 (m, 4H); 4.15 (q, J=7.2 Hz, 2H).

### EXAMPLE 57

### Ethyl 4-[3-(3-Chloro-phenyl)-1,1-dimethyl-prop-2-ynyl]-piperazine-1-carboxylic acid ethyl ester

1-Chloro-3-iodo-benzene (50.2 μL, 0.405 mmol) and 4-(1,1-dimethyl-prop-2-ynyl)-piperazine-1-carboxylic acid ethyl ester (113.4 mg, 0.446 mmol) were dissolved in triethylamine (2 mL) with stirring. Copper (I) iodide (7.7 mg, 0.0405 mmol), and bis(triphenylphosphine)palladium(II)chloride (14.26 mg, 0.0203 mmol) were added simultaneously to the reaction mixture. The reaction was stirred at room temperature overnight. The solution was diluted with dichloromethane and washed with water. The organic phase was dried, filtered and concentrated, then chromatography (50% ethyl acetate in hexanes) yielded the product (41 mg, 28%). ¹H NMR (300 MHz, CDCl₃): 1.26 (t, J=7.1 Hz, 3H); 1.47 (s, 6H); 2.66 (m, 4H); 3.53 (m, 4H); 4.14 (q, J=7.1 Hz, 2H); 7.25 (m, 3H); 7.39 (m, 1H).

### EXAMPLE 58

### 4-[3-(3-Chloro-phenyl)-1-ethyl-prop-2-ynyl]-piperazine-1-carboxylic acid methyl ester

1-[3-(3-chlorophenyl)-1-ethyl-prop-2-ynyl]-piperazine (40 mg, 0.152 mmol) was dissolved in dichloromethane (2 mL) and triethylamine (64 μL) was added with stirring. Methyl chloroformate (17.56 μL, 0.228 mmol) was added to the reaction mixture while keeping the reaction mixture at 0° C. After the addition the reaction was allowed to warm up to room temperature. When TLC analysis showed the reaction to be complete, the reaction mixture was diluted with dichloromethane and washed with water. The aqueous phase was re-extracted with dichloromethane and the combined organics were washed with brine, and then dried over Na₂SO₄ and concentrated. Chromatography (ethyl acetate, silica gel) yielded the product (40.3 mg, 82%). ¹H NMR (300 MHz, CDCl₃) δ=1.08 (t, J=7.4 Hz, 3H); 1.75 (m, 2H); 2.51 (m, 2H); 2.69 (m, 2H); 3.46 (t, J=7.5 Hz, 1H); 3.54 (m, 4H); 3.72 (s, 3H); 7.29 (m, 4H).

### EXAMPLE 59

### 4-[3-(3-Chloro-phenyl)-prop-2-ynyl]-piperazine-1-caroxylic acid 2-methoxy-ethyl ester

1-[3-(3-Chloro-phenyl)-prop-2-ynyl]-piperazine (30 mg, 0.128 mmol) was dissolved in dichloromethane (2 mL) and triethylamine (53.4 μL, 0.383 mmol) with stirring. (2-methoxy-ethyl)-chloroformate (22.1 μL, 0.1917 mmol) was added dropwise and the reaction mixture was stirred for 1 h. hen TLC analysis showed that the reaction was complete, it was diluted with dichloromethane and washed with water. The organic phase was dried (Na₂SO₄), filtered and concentrated under reduced pressure to an orange oil. Chromatography (SPE column, 50% ethyl acetate in hexanes) yielded the product (22.4 mg, 52%, yellowish oil). ¹H NMR (300 MHz, CDCl₃) δ=2.61 (m, 4H); 3.40 (s, 3H); 3.55 (s, 2H); 3.61 (m, 6H); 4.26 (t, J=4.6, 2H); 7.29 (m, 3H); 7.43 (m, 1H).

Pharmaceutical Examples

FLIPR Assay of Group I Receptor Antagonist Activity

For FLIPR analysis, cells were seeded on collagen coated clear bottom 96-well plates with black sides and analysis of [Ca²⁺]ᵢ mobilization was performed 24 hours following seeding. Cell cultures in the 96-well plates were loaded with a 4 μM solution of acetoxymethyl ester form of the fluorescent calcium indicator fluor-3 (Molecular Probes, Eugene, Oreg.) in 0.01% pluronic. All assays were performed in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4).

FLIPR experiments were done using a laser setting of 0.800 W and a 0.4 second CCD camera shutter speed with excitation and emission wavelengths of 488 nm and 562 nm, respectively. Each FLIPR experiment was initiated with 160 μL of buffer present in each well of the cell plate. A 40 μL addition from the antagonist plate was followed by a 50 μL addition from the agonist plate. After each addition the fluorescence signal was sampled 50 times at 1 second intervals followed by 3 samples at 5 second intervals. Responses were measured as the peak height of the response within the sample period.

EC₅₀/IC₅₀ determinations were made from data obtained from 8 point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

Measurement of Inositol Phosphate (IP3) Turnover in Intact Whole Cells

GHEK stably expressing the human mGluR5 receptor were seeded onto 24 well poly-L-lysine coated plates at 40×10⁴ cells/well in media containing 1 μCii/well [3H] myo-inositol. Cells were incubated overnight (16 h), then washed three times and incubated for 1 hour at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells were washed once in HEPES buffered saline and pre-incubated for 10 minutes in HEPES buffered saline containing 10 mM LiCl. Compounds (agonists) were added and incubated at 37° C. for 30 minutes. Antagonist activity was determined by pre-incubating test compounds for 15 minutes, then incubating in the presence of glutamate (80 μM) or DHPG (30 μM) for 30 minutes. The reaction was terminated by the addition of 0.5 ml perchloric acid (5%) on ice, with incubation at 4° C. for at least 30 minutes. Samples were collected in 15 ml Falcon tubes and inositol phosphates were separated using Dowex columns, as described below.

Assay For Inositol Phosphates Using Gravity-Fed Ion-Exchange Columns

Preparation of Ion-Exchange Columns

Ion-exchange resin (Dowex AG1-X8 formate form, 200-400 mesh, BIORAD) was washed three times with distilled water and stored at 4° C. 1.6 ml resin was added to each column and washed with 3 ml 2.5 mM HEPES, 0.5 mM EDTA, pH 7.4.

Sample Treatment

Samples were collected in 15 ml Falcon tubes and neutralized with 0.375 M HBEPES, 0.75 M KOH. 4 ml of HEPES/EDTA (2.5/0.5 mM, pH 7.4) were added to precipitate the potassium perchlorate. Supernatant was added to the prepared Dowex columns.

Inositol Phosphate Separation

Elute glycero phosphatidyl inositols with 8 ml 30 mM ammonium formate. Elute total inositol phosphates with 8 ml 700 mM ammonium formate/100 mM formic acid and collect eluate in scintillation vials. Count eluate mixed with 8 ml scintillant.

Screening for Compounds Active Against tlesr

Adult Labrador retrievers of both genders, trained to stand in a Pavlov sling, are used. Mucosa-to-skin esophagostomies are formed and the dogs are allowed to recover completely before any experiments are done.

Motility Measurement

In brief, after fasting for approximately 17 h with free supply of water, a multilumen sleeve/sidehole assembly (Dentsleeve, Adelaide, South Australia) is introduced through the esophagostomy to measure gastric, lower esophageal sphincter (LES) and esophageal pressures. The assembly is perfused with water using a low-compliance manometric perfusion pump (Dentsleeve, Adelaide, South Australia). An air-perfused tube is passed in the oral direction to measure swallows, and an antimony electrode monitored pH, 3 cm above the LES. All signals are amplified and acquired on a personal computer at 10 Hz.

When a baseline measurement free from fasting gastric/LES phase III motor activity has been obtained, placebo (0.9% NaCl) or test compound is administered intravenously (i.v., 0.5 ml/kg) in a foreleg vein. Ten min after i.v. administration, a nutrient meal (10% peptone, 5% D-glucose, 5% Intralipid, pH 3.0) is infused into the stomach through the central lumen of the assembly at 100 m/min to a final volume of 30 ml/kg. The infusion of the nutrient meal is followed by air infusion at a rate of 500 ml/min until an intragastric pressure of 10±1 mmHg is obtained. The pressure is then maintained at this level throughout the experiment using the infusion pump for further air infusion or for venting air from the stomach. The experimental time from start of nutrient infusion to end of air insufflation is 45 min. The procedure has been validated as a reliable means of triggering TLESRs.

TLESRs is defined as a decrease in lower esophageal sphincter pressure (with reference to intragastric pressure) at a rate of >1 mmHg/s. The relaxation should not be preceded by a pharyngeal signal <2s before its onset in which case the relaxation is classified as swallow-induced. The pressure difference between the LES and the stomach should be less than 2 mmHg, and the duration of the complete relaxation longer than 1 s.

Abbreviations

BSA Bovine Serum Albumin
CCD Charge Coupled Device
CRC Concentration Response Curve
DHPG 3,5-dihydroxyphenylglycine;
EDTA Ethylene Diamine Tetraacetic Acid
FLIPR Fluorometric Imaging Plate reader
GHEK GLAST-containing Human Embrionic Kidney
GLAST glutamate/aspartate transporter
HEPES 4-(2-hydroxyethyl)-1-piperazineethanesulfonic acid (buffer)
IP₃ inositol triphosphate
   Results

Typical IC₅₀ values as measured in the assays described above are 10 μM or less. In one aspect of the invention the IC₅₀ is below 2 μM. In another aspect of the invention the IC₅₀ is below 0.2 μM. In a further aspect of the invention the IC₅₀ is below 0.05 μM.

1. A compound according to formula I: 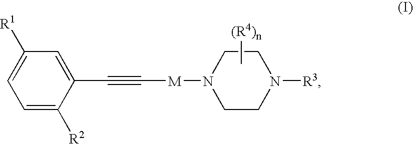
wherein
R¹ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;
R² is selected from the group consisting of hydrogen, hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵(CO)R⁶, OC₂₋₆alkylNR⁵(CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, SO₃R⁵ and a 5- or 6-membered ring containing atoms independently selected from the group consisting of C, N, O and S;
R³ is selected from the group consisting of:
H, C(O)OC₁₋₆alkylhalo, C(O)OC₁₋₆alkyl, C(O)OC₂₋₆alkenyl, C(O)OC₂₋₆alkynyl, C(O)OC₀₋₆alkylC₃₋₆cycloalkyl, C(O)OC₀₋₆alkylaryl, C(O)OC₁₋₆alkylOR⁵, C(O)OC₁₋₆alkyl(CO)R⁵, C(O)OC₁₋₆alkylCO₂R⁵, C(O)OC₁₋₆alkylcyano, C(O)OC₀₋₆alkylNR⁵R⁶, C(O)OC₁₋₆alkyl(CO)NR⁵R⁶, C(O)OC₂₋₆alkylNR⁵(CO)R⁶, C(O)C₁₋₆alkylNR⁵(CO)NR⁵R⁶, C(O)OC₂₋₆alkylSR⁵, C(O)OC₁₋₆alkyl(SO)R⁵, C(O)OC₁₋₆alkylSO₂R⁵, C(O)OC₁₋₆alkyl(SO₂)NR⁵R⁶, C(O)OC₁₋₆alkylNR⁵(SO₂)R⁶, C(O)OC₂₋₆alkylNR⁵(SO₂)NR⁵R⁶, (CO)NR⁵R⁶, C(O)OC₁₋₆alkylNR⁵(CO)OR⁶, C(S)OC₁₋₆alkylhalo, C(S)OC₁₋₆alkyl, C(S)OC₂₋₆alkenyl, C(S)OC₂₋₆alkynyl, C(S)OC₀₋₆alkylC₃₋₆cycloalkyl, C(S)OC₀₋₆alkylaryl, C(S)OC₁₋₆alkylOR⁵, C(S)OC₁₋₆alkyl(CO)R⁵, C(S)OC₁₋₆alkylCO₂R⁵, C(S)OC₁₋₆alkylcyano, C(S)OC₀₋₆alkylNR⁵R⁶, C(S)OC₁₋₆alkyl(CO)NR⁵R⁶, C(S)OC₂₋₆alkylNR⁵(CO)R⁶, C(S)C₁₋₆alkylNR⁵(CO)NR⁵R⁶, C(S)OC₂₋₆alkylSR⁵, C(S)OC₁₋₆alkyl(SO)R⁵, C(S)OC₁₋₆alkylSO₂R⁵, C(S)OC₁₋₆alkyl(SO₂)NR⁵R⁶, C(S)OC₁₋₆alkylNR⁵(SO₂)R⁶, C(S)OC₂₋₆alkylNR⁵(SO₂)NR⁵R⁶, (CO)NR⁵R⁶, and C(S)OC₁₋₆alkylNR⁵(CO)OR⁶;
R⁴ is selected from the group consisting of hydroxy, halo, nitro, C₁₋₆alkylhalo, OC₁₋₆alkylhalo, C₁₋₆alkyl, OC₁₋₆alkyl, C₂₋₆alkenyl, OC₂₋₆alkenyl, C₂₋₆alkynyl, OC₂₋₆alkynyl, C₀₋₆alkylC₃₋₆cycloalkyl, OC₀₋₆alkylC₃₋₆cycloalkyl, C₀₋₆alkylaryl, OC₀₋₆alkylaryl, CHO, (CO)R⁵, O(CO)R⁵, O(CO)OR⁵, O(CN)OR⁵, C₁₋₆alkylOR⁵, OC₂₋₆alkylOR⁵, C₁₋₆alkyl(CO)R⁵, OC₁₋₆alkyl(CO)R⁵, C₀₋₆alkylCO₂R⁵, OC₁₋₆alkylCO₂R⁵, C₀₋₆alkylcyano, OC₂₋₆alkylcyano, C₀₋₆alkylNR⁵R⁶, OC₂₋₆alkylNR⁵R⁶, C₁₋₆alkyl(CO)NR⁵R⁶, OC₁₋₆alkyl(CO)NR⁵R⁶, C₀₋₆alkylNR⁵ (CO)R⁶, OC₂₋₆alkylNR⁵ (CO)R⁶, C₀₋₆alkylNR⁵(CO)NR⁵R⁶, C₀₋₆alkylSR⁵, OC₂₋₆alkylSR⁵, C₀₋₆alkyl(SO)R⁵, OC₂₋₆alkyl(SO)R⁵, C₀₋₆alkylSO₂R⁵, OC₂₋₆alkylSO₂R⁵, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, C₀₋₆alkylNR⁵(SO₂)NR⁵R⁶, OC₂₋₆alkylNR⁵(SO₂)NR⁵R⁶, (CO)NR⁵R⁶, O(CO)NR⁵R⁶, NR⁵OR⁶, C₀₋₆alkylNR⁵(CO)OR⁶, OC₂₋₆alkylNR⁵(CO)OR⁶, ═NR⁵, ═NOR⁵, ═O, ═S, SO₃R⁵ and a 5- or 6-membered ring containing atoms independently selected from the group consisting of C, N, O and S;
M is selected from the group consisting of ═O, (CR⁵R⁶)ₘ and (CR⁵R⁶)ₘC(O);
R⁵ and R⁶ are independently selected from the group consisting of hydrogen, C₁₋₆alkyl, OC₁₋₆alkyl, C₃₋₇cycloalkyl, OC₃₋₇cycloalkyl, C₁₋₆alkylaryl, OC₁₋₆alkylaryl, aryl, and heteroaryl;
any C₁₋₆alkyl, aryl or heteroaryl defined under R¹, R², R³, R⁴, R⁵ and R⁶ may be substituted by one or more A;
A is selected from the group consisting of hydrogen, hydroxy, halo, nitro, oxo, C₀₋₆alkylcyano, C₀₋₄alkylC₃₋₆cycloalkyl, C₁₋₆alkyl, C₁₋₆alkylhalo, OC₁₋₆alkylhalo, C₂₋₆alkenyl, C₀₋₃alkylaryl, C₀₋₆alkylOR⁵, OC₂₋₆alkylOR⁵, C₁₋₆alkylSR⁵, OC₂₋₆alkylSR⁵, (CO)R⁵, O(CO)R⁵, OC₂₋₆alkylcyano, OC₁₋₆alkylCO₂R⁵, O(CO)OR⁵, OC₁₋₆alkyl(CO)R⁵, C₁₋₆alkyl(CO)R⁵, NR⁵OR⁶, C₁₋₆alkylNR⁵R⁶, OC₂₋₆alkylNR⁵R⁶, C₀₋₆alkyl(CO)NR⁵R⁶, OC₁₋₆alkyl(CO)NR⁵R⁶, OC₂₋₆alkylNR⁵(CO)R⁶, C₀₋₆alkylNR⁵(CO)R⁶, C₀₋₆alkylNR⁵(CO)NR⁵R⁶, O(CO)NR⁵R⁶, C₀₋₆alkyl(SO₂)NR⁵R⁶, OC₂₋₆alkyl(SO₂)NR⁵R⁶, C₀₋₆alkylNR⁵(SO₂)R⁶, OC₂₋₆alkylNR⁵(SO₂)R⁶, SO₃R⁵, C₁₋₆alkylNR⁵(SO₂)NR⁵R⁶, OC₂₋₆alkyl(SO₂)R⁵, C₀₋₆alkyl(SO₂)R⁵, C₀₋₆alkyl(SO)R⁵, OC₂₋₆alkyl(SO)R⁵ and a 5- or 6-membered ring containing one or more atoms independently selected from the group consisting of C, N, O and S;
m is 1, 2, or 3;
n is an integer between 0 and 8, inclusive; or
a pharmaceutically acceptable salt or hydrate thereof.

2. The compound according to claim 1, wherein n is 0.

3. The compound according to claim 2, wherein R³ is selected from the group consisting of:
C(O)OC₁₋₆alkylhalo, C(O)OC₁₋₆alkyl, C(O)OC₂₋₆alkenyl, C(O)OC₂₋₆alkynyl, C(O)OC₀₋₆alkylC₃₋₆cycloalkyl, C(O)OC₀₋₆alkylaryl, C(O)OC₁₋₆alkylOR⁵, C(O)OC₁₋₆alkyl(CO)R⁵, C(O)OC₁₋₆alkylCO₂R⁵, C(O)OC₁₋₆alkylcyano, C(O)OC₀₋₆alkylNR⁵R⁶, C(O)OC₁₋₆alkyl(CO)NR⁵R⁶, C(O)OC₂₋₆alkylNR⁵(CO)R⁶, C(O)C₁₋₆alkylNR⁵(CO)NR⁵R⁶, C(O)OC₂₋₆alkylSR⁵, C(O)OC₁₋₆alkyl(SO)R⁵, C(O)OC₁₋₆alkylSO₂R⁵, C(O)OC₁₋₆alkyl(SO₂)NR⁵R⁶, C(O)OC₁₋₆alkylNR⁵(SO₂)R⁶, C(O)OC₂₋₆alkylNR⁵(SO₂)NR⁵R⁶, (CO)NR⁵R⁶, and C(O)OC₁₋₆alkylNR⁵(CO)OR⁶.

4. The compound according to claim 3, wherein R³ is selected from the group consisting of C(O)OC₁₋₆alkyl, C(O)OC₀₋₆alkylaryl, C(O)OC₁₋₆alkylOR⁵, and (CO)NR⁵R⁶.

5. The compound according to claim 2, wherein R² is hydrogen or fluoro.

6. The compound according to claim 5, wherein M is CR⁵R⁶.

7. The compound according to claim 6, wherein R⁶ in M is H.

8. The compound according to claim 7, wherein R⁵ in M is selected from hydrogen, C₁₋₆alkyl, C₃₋₇cycloalkyl, C₁₋₆alkylaryl, aryl, and heteroaryl.

9. The compound according to claim 8, wherein R⁵ is C₁₋₆alkyl.

10. The compound according to claim 8, wherein R⁵ is C₃₋₇cycloalkyl.

11. The compound according to claim 8, wherein R⁵ is heteroaryl.

12. The compound according to claim 11, wherein heteroaryl is selected from the group consisting of 2-, 3-, and 4-pyridyl; 2- and 3-thienyl; and 2- and 3-furanyl.

13. The compound according to claim 8, wherein R⁵ is aryl.

14. The compound according to claim 13, wherein aryl is phenyl.

15. The compound according to claim 1, selected from the group consisting of:
4-[3-(3-Chloro-phenyl)-prop-2-ynyl]-piperazine-1-carboxylic acid ethyl ester,
4-(3-Phenyl-prop-2-ynyl)-piperazine-1-carboxylic acid ethyl ester,
4-[3-(3-Cyano-phenyl)-prop-2-ynyl]-piperazine-1-carboxylic acid ethyl ester,
4-(3-m-Tolyl-prop-2-ynyl)-piperazine-1-carboxylic acid ethyl ester,
4-[3-(3-Methoxy-phenyl)-prop-2-ynyl]-piperazine-1-carboxylic acid ethyl ester,
4-[3-(5-Cyano-2-fluoro-phenyl)-prop-2-ynyl]-piperazine-1-carboxylic acid ethyl ester,
4-[3-(2-Fluoro-5-methyl-phenyl)-prop-2-ynyl]-piperazine-1-carboxylic acid ethyl ester,
4-[3-(5-Chloro-2-fluoro-phenyl)-prop-2-ynyl]-piperazine-1-carboxylic acid ethyl ester,
4-[3-(3-Chloro-phenyl)-1-methyl-prop-2-ynyl]-piperazine-1-carboxylic acid ethyl ester,
4-[3-(3-Chloro-phenyl)-1-ethyl-prop-2-ynyl]-piperazine-1-carboxylic acid ethyl ester,
4-[3-(3-Chloro-phenyl)-1-isopropyl-prop-2-ynyl]-piperazine-1-carboxylic acid ethyl ester,
4-[1-tert-Butyl-3-(3-chloro-phenyl)-prop-2-ynyl]-piperazine-1-carboxylic acid ethyl ester,
4-[3-(3-Chloro-phenyl)-1-phenyl-prop-2-ynyl]-piperazine-1-carboxylic acid ethyl ester,
4-[1-(3-Chloro-phenylethynyl)-butyl]-piperazine-1-carboxylic acid ethyl ester,
4-[1-(3-Chloro-phenylethynyl)-3-methyl-butyl]-piperazine-1-carboxylic acid ethyl ester,
4-[1-Benzyloxymethyl-3-(3-chloro-phenyl)-prop-2-ynyl]-piperazine-1-carboxylic acid ethyl ester,
4-[3-(3-Chloro-phenyl)-1-cyclopropyl-prop-2-ynyl]-piperazine-1-carboxylic acid ethyl ester,
4-[1-(3-Chloro-phenylethynyl)-pentyl]-piperazine-1-carboxylic acid ethyl ester,
4-[3-(3-Chloro-phenyl)-1-thiophen-2-yl-prop-2-ynyl]-piperazine-1-carboxylic acid ethyl ester,
4-[3-(3-Chloro-phenyl)-1-thiophen-3-yl-prop-2-ynyl]-piperazine-1-carboxylic acid ethyl ester,
4-[3-(3-Chloro-phenyl)-1-furan-2-yl-prop-2-ynyl]-piperazine-1-carboxylic acid ethyl ester,
4-[3-(3-Chloro-phenyl)-1-ethyl-prop-2-ynyl]-piperazine-1-carboxylic acid tert-butyl ester,
1-[3-(3-Chloro-phenyl)-1-ethyl-prop-2-ynyl]-piperazine,
4-[3-(3-Chloro-phenyl)-1-ethyl-prop-2-ynyl]-piperazine-1-carboxylic acid isopropyl ester,
4-[3-(3-Chloro-phenyl)-1-ethyl-prop-2-ynyl]-piperazine-1-carboxylic acid propyl ester,
4-[3-(3-Chloro-phenyl)-1-ethyl-prop-2-ynyl]-piperazine-1-carboxylic acid isobutyl ester,
4-[3-(3-Chloro-phenyl)-1-ethyl-prop-2-ynyl]-piperazine-1-carboxylic acid butyl ester,
4-[3-(3-Chloro-phenyl)-1-ethyl-prop-2-ynyl]-piperazine-1-carboxylic acid 2,2-dimethyl-propyl ester,
4-[3-(3-Chloro-phenyl)-1-ethyl-prop-2-ynyl]-piperazine-1-carboxylic acid pentyl ester,
4-[3-(3-Chloro-phenyl)-1-ethyl-prop-2-ynyl]-piperazine-1-carboxylic acid 2-methoxy-ethyl ester,
4-[3-(3-Chloro-phenyl)-1-ethyl-prop-2-ynyl]-piperazine-1-carboxylic acid phenyl ester,
4-[3-(3-Chloro-phenyl)-1-ethyl-prop-2-ynyl]-piperazine-1-carboxylic acid benzyl ester,
4-[3-(3-Chloro-phenyl)-1-pyridin-3-yl-prop-2-ynyl]-piperazine-1-carboxylic acid ethyl ester,
4-[3-(3-Chloro-phenyl)-1-(2,4-difluoro-phenyl)-prop-2-ynyl]-piperazine-1-carboxylic acid ethyl ester,
4-[3-(3-Chloro-phenyl)-1-(2-methoxy-phenyl)-prop-2-ynyl]-piperazine-1-carboxylic acid ethyl ester,
4-[3-(3-Chloro-phenyl)-1-(2-chloro-phenyl)-prop-2-ynyl]-piperazine-1-carboxylic acid ethyl ester,
4-[3-(3-Chloro-phenyl)-1-o-tolyl-prop-2-ynyl]-piperazine-1-carboxylic acid ethyl ester,
4-[3-(3-Chloro-phenyl)-1-m-tolyl-prop-2-ynyl]-piperazine-1-carboxylic acid ethyl ester,
4-[3-(3-Chloro-phenyl)-1-(6-methoxy-pyridin-3-yl)-prop-2-ynyl]-piperazine-1-carboxylic acid ethyl ester,
4-[3-(3-Chloro-phenyl)-1-(2-chloro-pyridin-3-yl)-prop-2-ynyl]-piperazine-1-carboxylic acid ethyl ester,
Ethyl 4-[3-(5-chloro-2-fluorophenyl)-1-ethylprop-2-yn-1-yl]piperazine-1-carboxylate
Ethyl 4-[3-(3-chlorophenyl)-1-(5-methyl-2-furyl)prop-2-yn-1-yl]piperazine-1-carboxylate
Ethyl 4-{3-(3-chlorophenyl)-1-[5-(methoxycarbonyl)-2-furyl]prop-2-yn-1-yl}piperazine-1-carboxylate
2,2,2-Trifluoroethyl 4-[3-(3-chlorophenyl)-1-(2-furyl)prop-2-yn-1-yl]piperazine-1-carboxylate
Ethyl 4-{3-(3-chlorophenyl)-1-[5-(hydroxymethyl)-2-furyl]prop-2-yn-1-yl}piperazine-1-carboxylate
Ethyl (3S)-4-[(1R)-3-(3-chlorophenyl)-1-(2-furyl)prop-2-yn-1-yl]-3-methylpiperazine-1-carboxylate
Ethyl (3S)-4-[(1S)-3-(3-chlorophenyl)-1-(2-furyl)prop-2-yn-1-yl]-3-methylpiperazine-1-carboxylate
Ethyl (3R)-4-[(1S)-3-(3-chlorophenyl)-1-ethylprop-2-yn-1-yl]-3-methylpiperazine-1-carboxylate
Ethyl (3R)-4-[(1R)-3-(3-chlorophenyl)-1-(2-furyl)prop-2-yn-1-yl]-3-methylpiperazine-1-carboxylate
Ethyl (3R)-4-[(1R)-3-(3-chlorophenyl)-1-ethylprop-2-yn-1-yl]-3-methylpiperazine-1-carboxylate
Ethyl (3S)-4-[(1S)-3-(3-chlorophenyl)-1-ethylprop-2-yn-1-yl]-3-methylpiperazine-1-carboxylate
Ethyl (3S)-4-[(1R)-3-(3-chlorophenyl)-1-methylprop-2-yn-1-yl]-3-methylpiperazine-1-carboxylate
4-[3-(3-Chloro-phenyl)-prop-2-ynyl]-piperazine-1-carboxylic acid tert-butyl ester
4-[1-(Tert-Butoxycarbonylamino-methyl)-3-(3-chloro-phenyl)-prop-2-ynyl]-piperazine-1-carboxylic acid ethyl ester
4-[3-(3-Chloro-phenyl)-1-triisopropylsilyloxymethyl-prop-2-ynyl]-piperazine-1-carboxylic acid ethyl ester
Ethyl 4-[3-(3-chlorophenyl)-1-(ethoxymethyl)prop-2-yn-1-yl]piperazine-1-carboxylate
4-[1-Aminomethyl)-3-(3-chloro-phenyl)-prop-2-ynyl]-piperazine-1-carboxylic acid ethyl ester
4-[3-(3-Chloro-phenyl)-1-hydroxymethyl-prop-2-ynyl]-piperazine-1-carboxylic acid ethyl ester
4-[3-(3-Chloro-phenyl)-1-methoxymethyl-prop-2-ynyl]-piperazine-1-carboxylic acid ethyl ester
4-(3-Phenyl-propynoyl)-piperazine-1-carboxylic acid ethyl ester
Ethyl 4-[3-(3-Chloro-phenyl)-1,1-dimethyl-prop-2-ynyl]-piperazine-1-carboxylic acid ethyl ester
4-[3-(3-Chloro-phenyl)-1-ethyl-prop-2-ynyl]-piperazine-1-carboxylic acid methyl ester 4-[3-(3-Chloro-phenyl)-prop-2-ynyl]-piperazine-1-caroxylic acid 2-methoxy-ethyl ester, and
pharmaceutically acceptable salts or hydrates thereof.

16. A pharmaceutical composition comprising as active ingredient a therapeutically effective amount of the compound according to claim 1, in association with one or more pharmaceutically acceptable diluents, excipients and/or inert carriers.

17. The pharmaceutical composition according to claim 16, for use in the treatment of mGluR 5 mediated disorders.

18. The compound according to claim 1, for use in therapy.

19. The compound according to claim 1, for use in treatment of mGluR 5 mediated disorders.

20. Use of the compound according to claim 1, in the manufacture of a medicament for the treatment of mGluR 5 mediated disorders.

21. A method of treatment of mGluR 5 mediated disorders, comprising administering to a mammal a therapeutically effective amount of the compound according to claim 1.

22. The method according to claim 21, wherein the mammal is a human.

23. The method according to claim 21, wherein the disorders are neurological disorders.

24. The method according to claim 21, wherein the disorders are psychiatric disorders.

25. The method according to claim 21, wherein the disorders are chronic and acute pain disorders.

26. The method according to claim 21, wherein the disorders are gastrointestinal disorders.

27. A method for inhibiting activation of mGluR 5 receptors, comprising treating a cell containing said receptor with an effective amount of the compound according to claim 1.
